(19) United States
(12) Patent Application Publication
(10) Pub. No.: US 2007/0190065 A1
(43) Pub. Date: Aug. 16, 2007

(54) Nucleic acids, polypeptides, methods of expression, and immunogenic compositions associated with SARS corona virus spike protein

(76) Inventors: Ralf Altmeyer, Singapore (SG); Beatrice Nal-Rogier, Hong Kong (CN); Cheman Chan, Hong Kong (CN); Francois Kien, Hong Kong (CN); Yiu Wing Kam, Hong Kong (CN); Yu Lam Siu, Hong Kong (CN); Kong San Tse, Hong Kong (CN); Isabelle Staropoli, Paris (FR); Jean-Claude Manuguerra, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/635,822

(22) Filed: Dec. 4, 2006

Related U.S. Application Data
(63) Continuation of application No. PCT/EP05/06512, filed on Jun. 3, 2005.

(51) Int. Cl. A61K 39/42; A61K 39/215; C12Q 1/70; C07H 21/04; C12P 21/06; C07K 14/165; C12N 15/86

(52) U.S. Cl. 424/159.1; 435/5; 435/69.1; 435/456; 435/325; 530/350; 530/388.3; 536/23.72; 424/221.1

(57) ABSTRACT

Nucleic acid molecules, polypeptides, immunogenic compositions, vaccines, and methods of making and using the nucleotides and encoded polypeptides associated with the Spike protein of SARS Corona Virus (SARS CoV) are disclosed.

### RELATED APPLICATIONS

This application claims the benefit of U.S. application Ser. No. 10/860,641, filed Jun. 4, 2004, and U.S. Provisional Application No. 60/578,348, filed Jun. 10, 2004, all of which are incorporated herein by reference. A Request for Conversion to Provisional Application was filed in U.S. application Ser. No. 10/860,641 on May 23, 2005.

### FIELD OF THE INVENTION

The invention is directed to purified and isolated nucleic acids, polypeptides, purified and isolated polypeptides, the nucleic acids encoding such polypeptides, processes for production of recombinant forms of such polypeptides, antibodies generated against these polypeptides, and the use of such nucleic acids and polypeptides in diagnostic methods, kits, immunogenic compositions, vaccines, or antiviral therapy.

### BACKGROUND OF THE INVENTION

A new infectious disease, known as severe acute respiratory syndrome (SARS), appeared in Guangdong province of southern China in 2002. SARS spread to 29 countries, affected a reported 8,098 people, and left 774 patients dead. (Stadler et al.) Although the SARS epidemic was contained by aggressive quarantine measures, there is no information on when, or if, SARS will re-emerge in the human population.

SARS is mainly characterized by flu-like symptoms, including high fevers exceeding 100.4° F., myalagia, dry nonproductive dyspnea, lymphopaenia, and infiltrate on chest radiography. (Stadler et al.) In 38% of all cases, the resulting pneumonia led to acute breathing problems requiring artificial respirators. The overall mortality rate was about 10%, but varied profoundly with age, as SARS appeared to be milder in the pediatric age group while the mortality rate in the elderly was as high as 50%.

SARS is caused by a previously unknown coronavirus (CoV), a diverse group of large, enveloped viruses that cause respiratory and enteric disease in humans and animals. SARS CoV was isolated from FRhK-4 and Vero E6 cells that were inoculated with clinical specimens from patients, and macaques inoculated with this virus developed symptoms similar to those observed in human cases of SARS. To date, over 30 different SARS CoV have been isolated and sequenced.

SARS CoV contains an RNA genome of about 30 kB (Accession No. AY310120), and shares many characteristic features of coronaviruses. Nucleotides 1-72 contain a predicted RNA leader sequence preceding an untranslated region (UTR) spanning 192 nucleotides. Two overlapping open reading frames, which encompass approximately two-thirds of the genome (nucleotides 265-21485) are down stream of the UTR, and encode proteinases as well as the proteins required for replication and transcription (for a review see Stadler et al., 2004). The remaining 3′ part of the genome encodes four structural proteins that are arranged in the same order in all CoV: Spike, Envelope, Membrane glycoprotein, and Nucleocapsid protein. The structural protein region of the SARS CoV genome also encodes additional non-structural proteins known as ‘accessory genes’. Although the overall organization of the SARS CoV genome is similar to other coronaviruses, the amino acid conservation of the encoded proteins is usually low.

The Spike protein forms large surface projections that are characteristic of coronaviruses. Spike is heavily glycosylated and has 1,255 amino acids, containing an amino-terminal bulbous head adjacent to a stem, a single transmembrane region, and a short cytoplasmic tail (See Stadler et al.).

Although β-interferon has been reported to interfere with the replication of the SARS virus in vitro, no licensed drug or vaccine is available. Moreover, large-scale screening of existing antivirals or big chemical libraries for potential replication inhibitors has not been very successful. It is also virtually impossible to confirm a SARS diagnosis in the primary care setting, as the sensitivity and specificity of available tests varies with time from onset of contact or symptoms (See Rainer et al.). At present, there are no easy, rapid, accurate tests for diagnosing SARS during the first week of illness, and none that will give a result within hours of sampling. For these reasons, there is considerable need for the development of a detailed understanding of SARS CoV proteins. Such an understanding can provide effective means to treat or control the infection, as well as aid in the diagnosis of SARS CoV infection in humans.

### SUMMARY OF THE INVENTION

Accordingly, this invention aids in fulfilling these needs in the art. The invention encompasses a purified nucleic acid molecule comprising the DNA sequences of SEQ ID NO: 2, SEQ ID NO: 3, or SEQ ID NO: 6. The invention also encompasses nucleic acid molecules complementary to these sequences, such as fully complementary sequences.

The invention includes double-stranded nucleic acid molecules comprising the DNA sequence of SEQ ID NO: 2, SEQ ID NO: 3, or SEQ ID NO: 6 and purified nucleic acid molecules encoding the amino acid sequence of SEQ ID NO: 4 or SEQ ID NO: 7. Both single-stranded and double-stranded RNA and DNA nucleic acid molecules are encompassed by the invention. These molecules can be used to detect both single-stranded and double-stranded RNA and DNA encompassed by the invention. A double-stranded DNA probe allows the detection of nucleic acid molecules equivalent to either strand of the nucleic acid molecule.

Purified nucleic acid molecules that hybridize to a denatured, double-stranded DNA comprising the DNA sequence of SEQ ID NO: 2, SEQ ID NO: 3, or SEQ ID NO: 6 or a purified nucleic acid molecule encoding the amino acid sequence of SEQ ID NO: 4 or SEQ ID NO: 7 under conditions of high stringency are encompassed by the invention.

The invention further encompasses purified nucleic acid molecules derived by in vitro mutagenesis from SEQ ID NOS: 1-3 & 6. In vitro mutagenesis includes numerous techniques known in the art including, but not limited to, site-directed mutagenesis, random mutagenesis, and in vitro nucleic acid synthesis.

The nucleic acid molecules of the invention, which include DNA and RNA, are referred to herein as “Spike nucleic acids” or “Spike DNA”, and the amino acids encoded by these molecules are referred to herein as “Spike polypeptides” or “Spike protein.”

The invention also encompasses purified nucleic acid molecules degenerate from SEQ ID NOS: 1-3 & 6 as a result of the genetic code, purified nucleic acid molecules, which are allelic variants of Spike nucleic acids or a species homolog of Spike nucleic acids.

The invention encompasses purified nucleic acids that show increased expression of Spike protein as compared to SEQ ID NO: 1.

The invention also encompasses purified nucleic acids that show increased expression of Spike protein as compared to SEQ ID NO: 1, wherein at least one negative cis-acting signal has been substituted without changing the sequence of the encoded protein. Negative cis-acting signals as encompassed by the invention include, but are not limited to, AU-rich RNA instability motifs, repeating sequences, secondary stretches, splice donor and acceptor sites, and internal poly(A) sites.

The invention also encompasses purified nucleic acid molecules that show increased expression of Spike protein as compared to SEQ ID NO: 1, wherein expression is increased through the addition of expression enhancing sequences. Expression enhancing sequences include, but are not limited to, Kozak consensus sequence upstream of the starting ATG, as well as additional stop codons.

A skilled artisan will know the suitable placement of the Kozak consensus sequence based on the prior art.

The invention also encompasses purified nucleic acid molecules that show increased expression of Spike protein as compared to SEQ ID NO: 1, wherein codon usage has been optimized to the bias of Cricetulus griseus.

The invention also encompasses purified nucleic acid molecules that show increased expression of Spike protein as compared to SEQ ID NO: 1, wherein the portion of the purified nucleic acid molecule encoding Spike protein comprises at least about a 10 percent increase in the percentage GC-content as compared to SEQ ID NO: 1, and wherein regions of very high (>80%) or very low (<30%) GC content have been avoided where possible.

The invention also encompasses purified nucleic acid molecules that show increased expression of Spike protein as compared to SEQ ID NO: 1, wherein the substitution of at least one negative cis-acting signal and wherein the at least one additional expression enhancing sequence does not include the following: internal TATA-boxes, chi-sites, and ribosomal entry sites; AT-rich or GC-rich sequence stretches; repeat sequences and RNA secondary structures; and splice donor and acceptor sites.

The invention also encompasses purified polypeptides encoded by these nucleic acid molecules, including glycosylated and non-glycosylated forms of the purified polypeptide.

The invention also encompasses recombinant vectors that direct the expression of these nucleic acid molecules and host cells transformed or transfected with these vectors.

Purified polyclonal or monoclonal antibodies that bind to Spike polypeptides are encompassed by the invention, as are neutralizing antibodies.

The invention further encompasses methods for the production of Spike polypeptides, including culturing a host cell under conditions for promoting expression, and recovering the polypeptide from the culture medium. Especially, the expression of Spike polypeptides in animal cells is encompassed by the invention.

The invention also encompasses labeled Spike polypeptides. Preferably, the labeled polypeptides are in purified form. It is also preferred that the unlabeled or labeled polypeptide is capable of being immunologically recognized by human body fluid containing antibodies to Spike polypeptide. The polypeptides can be labeled, for example, with an immunoassay label selected from the group consisting of radioactive, enzymatic, fluorescent, chemiluminescent labels, and chromophores.

Immunological complexes between the Spike polypeptides of the invention and antibodies recognizing the polypeptides are also provided. The immunological complexes can be labeled with an immunoassay label selected from the group consisting of radioactive, enzymatic, fluorescent, chemiluminescent labels, and chromophores.

Furthermore, this invention provides a method for detecting infection by SARS CoV. The method comprises providing a composition comprising a biological material suspected of being infected with SARS CoV, and assaying for the presence of Spike polypeptide of SARS CoV. The polypeptides are typically assayed by electrophoresis or by immunoassay with antibodies that are immunologically reactive with the Spike polypeptides of the invention.

This invention also provides an in vitro diagnostic method for the detection of the presence or absence of antibodies, which bind to an antigen comprising the Spike polypeptides of the invention. The method comprises contacting the antigen with a biological fluid for a time and under conditions sufficient for the antigen and antibodies in the biological fluid to form an antigen-antibody complex, and then detecting the formation of the complex. The detecting step can further comprise measuring the formation of the antigen-antibody complex. The formation of the antigen-antibody complex is preferably measured by immunoassay based on Western blot technique, ELISA (enzyme linked immunosorbent assay), FACS, indirect immunofluorescent assay, or immunoprecipitation assay.

The invention also encompasses a diagnostic kit for the detection of the presence or absence of antibodies, which bind to the Spike polypeptide of the invention, contains antigen comprising the Spike polypeptide, and means for detecting the formation of immune complex between the antigen and antibodies. The antigens and the means are present in an amount sufficient to perform the detection.

This invention also provides an immunogenic composition comprising a Spike polypeptide of the invention or a mixture thereof in an amount sufficient to induce an immunogenic or protective response in vivo, in association with a pharmaceutically acceptable carrier therefor. The immunogenic composition may contain an Alum adjuvant. A vaccine composition of the invention comprises a neutralizing amount of the Spike polypeptide and a pharmaceutically acceptable carrier therefor.

The polypeptides of this invention are thus useful as a portion of a diagnostic composition for detecting the presence of antibodies to antigenic proteins associated with SARS CoV.

In addition, the Spike polypeptides can be used to raise antibodies for detecting the presence of antigenic proteins associated with SARS CoV.

The polypeptides of the invention can also be employed to raise neutralizing antibodies that either inactivate the virus, reduce the viability of the virus in vivo, or inhibit or prevent viral replication. The ability to elicit virus-neutralizing antibodies is especially important when the polypeptides of the invention are used in immunizing or vaccinating compositions to activate the B-cell arm of the immune response or induce a cytotoxic T lymphocyte response (CTL) in the recipient host.

The present invention also pertains to vaccine compositions for immunizing humans and mammals against SARS CoV, comprising an immunogenic composition as described above in combination with one or more pharmaceutically compatible excipients (such as, for example, saline buffer), and optionally in combination with at least one adjuvant such as aluminum hydroxide or a compound belonging to the muramyl peptide family.

This invention also encompasses a method for detecting the presence or absence of SARS CoV comprising:

(1) contacting a sample suspected of containing viral genetic material of SARS CoV with at least one nucleotide probe, and

(2) detecting hybridization between the nucleotide probe and the viral genetic material in the sample,

wherein said nucleotide probe is complementary to the full-length sequence of the purified Spike nucleic acids of the invention.

Additional features and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the expression of Spike-HKU-PRC in transfected 293T cells. Lane 1 represents cells transfected with pcDNA-Spike-Pasteur, lane 2 represents cells transfected with pcDNA-HKU-PRC, lane 3 represents cells transfected with SFV-Spike-Pasteur-modif, lane 4 is empty, and lane 5 represents purified Spike from transfected BHK cells.

FIG. 2 shows the sequence in standard single letter abbreviations of the SARS CoV Spike protein with the Flag peptide sequence used for RNA and protein vaccination (SEQ ID NO: 5). The sequence corresponding to the SARS CoV Spike protein is shaded, while the sequence including the Flag peptide is underlined. The protein sequence was expressed in the Semliki (SFV) Forest Virus vector.

FIG. 3 is an SDS-PAGE of pulse-chase labeled SFV-Spike infected BHK cells following immunoprecipitation with M2 (Flag) antibody. Cells were harvested at the indicated time points after chase. The “*” denotes high-mannose N-glycan EndoH-sensitive Spike, the “O” represents complex N-glycan EndoH-resistant Spike, and the “#” represents high-mannose N-glycan EndoH-sensitive deglycosylated Spike.

FIG. 4 shows the plasma membrane expression of Spike in SFV Spike infected BHK cells. Spike protein labeling was realized with M2 antibody, while endoplasmic reticulum (ER) was stained with Erp72 monoclonal antibody.

FIG. 5 is a Western Blot analysis showing that the SARS CoV protein binds sACE2 receptor. M2-beads coated with Spike (lanes 1 and 4) or with BAP as a control (lanes 3 and 6) were incubated with sACE2 and run on an SDS-gel prior to Western blot with anti-ACE2 antibody or with Mab M2 as a control. While both Spike and BAP proteins are present in the reaction (lanes 4 and 6), only Spike binds to ACE2 (lanes 1 and 3).

FIG. 6 shows that mice immunized with the recombinant immunopurified SARS CoV Spike protein produce antibodies against recombinant Spike. Pooled mouse sera (n=5) were used at 1/100 fold dilution for the Western Blot. Lanes 1 and 2, respectively, represent Western blots using the preimmune sera from control (CTRL) and Spike vaccinated (VACC) animals. Lanes 3 and 4, respectively, represent Western blots using the day 34 sera of the CTRL and VACC groups, while lanes 5 and 6, respectively, represent Western blots using the day 42 sera of CTRL and VACC groups. Lane 7 represents human SARS patient serum, and lane 8 represents a commercial serum from a rabbit immunized with Spike protein at a 1/150 fold dilution.

FIG. 7 shows that mice immunized with the recombinant immunopurified SARS CoV Spike protein produce antibodies against recombinant Spike. Pooled mouse sera (n=5) were used at 1/50 fold dilution for FACS analysis. Human SARS patient serum is shown in the right panel as a control.

FIG. 8 shows that mice immunized with the recombinant immunopurified SARS CoV Spike protein produce antibodies against SARS CoV. Pooled mouse sera (n=5) were used at 1/50 fold dilution for immunofluorescence analysis on SARS CoV-infected or mock-infected FRHK4 cells.

FIGS. 9(A), 9(B), and 9(C) show the nucleic acid sequence of Spike-Pasteur (SEQ ID NO: 1). Each of the Spe I sites are underlined, and the nucleic residues replaced to form Spike-Pasteur-modif are shaded.

FIGS. 10(A), 10(B), and 10(C) show the nucleic acid sequence of Spike-Pasteur-modif (SEQ ID NO: 2). The mutations eliminating the Spe I sites from Spike-Pasteur are shaded.

FIGS. 11(A), 11(B), 11(C), 11(D), 11(E), and 11(F) show the nucleic acid sequence of Spike-HKU-PRC (SEQ ID NO: 3), as well as its complementary strand. The shaded nucleic acid sequence encodes Spike polypeptide. FIGS. 11(A), 11(B), 11(C), 11(D), 11(E), and 11(F) also show the amino acid sequence of Spike fused to the Flag peptide (SEQ ID NO: 4). Stop codons are labeled with asterisks.

FIGS. 12(A) and 12(B) show the optimized nucleic acid sequence (SEQ ID NO: 6) that encodes the SARS CoV Spike polypeptide within Spike-HKU-PRC. SEQ ID NO: 6 differs from SEQ ID NO: 3 in that it does not contain sequence that encodes the Flag peptide or upstream or downstream sequences.

FIG. 13 describes the sequence of the SARS CoV Spike polypeptide (SEQ ID NO: 7) encoded by Spike-HKU-PRC.

FIG. 14 describes a plasmid of the invention, labeled 040078pPCR-Script, which contains sequence encoding Spike-HKU-PRC. The synthetic gene 040078 was assembled from synthetic oligonucleotides. The fragment was cloned into pPCR-Script Amp (Stratagene, LaJolla, Calif., USA) using Kpnl and Sacl restriction sites.

FIG. 15 describes a plasmid of the invention, labeled 040086pcDNA3.1(+), also called 040078pcDNA3.1(+), which contains sequence encoding Spike-HKU-PRC cloned into pcDNA3.1(Invitrogen) using the BamHl restriction site.

FIG. 16 describes the purity of Spike protein used for vaccination, and shows an SDS-PAGE gel colored with silver stain. Samples included: (M) molecular weight marker; (1) 720 ng S-protein; (2) 360 ng S-protein; (3)180 ng S-protein; and (4) 90 ng S-protein. Molecular weights are indicated and the positions of complex glycosylated (upper arrow) and high-mannose (lower arrow) monomeric Spike protein are indicated by arrows.

FIG. 17 shows an enhanced serum antibody response in animals immunized with TriSpike+Alum. Sera from vaccinated mice were analyzed for reactivity with TriSpike. (A) A high-titer neutralizing SARS patient serum, a rabbit serum against S1, and M2 monoclonal antibody against the FLAG peptide were used as controls. Western Blot analysis of pooled sera from mice (n=3) immunized with TriSpike with (group A) or without (group B) Alum adjuvant. Sera were collected at indicated time points and used at 1/1000 dilution for Western Blot analysis. All sera were reacted with FLAG-tagged control protein (BAP-FLAG) to assess antibody production against the FLAG tag. Immune complexes were detected with HRP-conjugated goat anti-mouse, human, or rabbit IgG polyclonal antibody. (B) Serum from vaccinated mice were analysed for neutralizing activity against SARS CoV infection on FRhk4 cells in vitro. The neutralizing activity of serum from mice vaccinated with TriSpike alone dropped rapidly (from day 49 to 87), but mice vaccinated with TriSpike+Alum remained stable within the same period of comparison.

FIG. 18 shows the induction of a mucosal immune response in TriSpike+Alum vaccinated mice. Fecal and nasal lavage samples from immunized mice (TriSpike or TriSpike+Alum) were collected and analysed for reactivity with TriSpike (A-B). M2 monoclonal antibody against the FLAG peptide was used as a control. (A) Fecal samples from vaccinated mice were collected at day 44 and used at 1/500 dilution for Western Blot analysis. Immune complexes were detected with HRP-conjugated goat anti-mouse IgG or IgA polyclonal antibody. (B) describes the same experiment as (A), except that Western Blot analysis was performed with pooled nasal lavage samples from vaccinated mice collected at day 65. Nasal lavage samples were used at 1/25 dilution for Western Blot analysis. (C) Fecal samples from vaccinated mice were collected and analyzed for neutralizing activity against SARS CoV infection on FRhk4 cells in vitro. Weak neutralizing activity was detected after the third immunization only. Nasal lavage samples from immunized mice were analysed but no observable level of neutralizing activity obtained in vitro.

FIG. 19 shows the immunogenicity of TriSpike in Golden Syrian hamster. Sera from hamsters vaccinated with indicated concentrations of TriSpike+Alum and control hamsters were analyzed for reactivity with TriSpike and neutralization. (A) Reactivity of immune sera with native TriSpike protein using FACS analysis. Sera, diluted 1/100, from hamster immunized subcutaneously with 2, 10, or 20 μg of TriSpike (on day 0, 21 and 42) were reacted with live BHK-21 cells expressing TriSpike at the plasma membrane. Immune complexes were identified using FITC-conjugated goat anti-hamster IgG polyclonal antibody. Results are expressed as MFI (mean fluorescence intensity) values. The MFI value reached the maximum after the second immunization (post-dose 2) and remained stable after the third immunization (post-dose 3). (B) Neutralizing activity was obtained in a SARS CoV microneutralization assay (100TCID50/well final) on FRhk4 cells.

### DETAILED DESCRIPTION OF THE INVENTION

Optimized DNA sequences for increased expression of the Spike protein of the SARS CoV have been discovered, including Spike-Pasteur-modif (SEQ ID NO: 2) and Spike-HKU-PRC (SEQ ID NOS: 3 & 6).

Nucleic acid sequences within the scope of the invention include isolated DNA and RNA sequences that hybridize to SEQ ID NOS: 2, 3 & 6 herein under conditions of moderate or severe stringency, and which encode Spike polypeptides. As used herein, conditions of moderate stringency, as known to those having ordinary skill in the art, and as defined by Sambrook et al. Molecular Cloning: A Laboratory Manual, 2 ed. Vol. 1, pp. 1.101-104, Cold Spring Harbor Laboratory Press, (1989), include use of a prewashing solution for the nitrocellulose filters 5×SSC, 0.5% SDS, 1.0 mM EDTA (pH 8.0), hybridization conditions of 50% formamide, 6×SSC at 42EC (or other similar hybridization solution, such as Stark's solution, in 50% fornamide at 42EC), and washing conditions of about 60EC, 0.5×SSC, 0.1% SDS. Conditions of high stringency are defined as hybridization conditions as above, and with washing at 68EC, 0.2×SSC, 0.1% SDS. The skilled artisan will recognize that the temperature and wash solution salt concentration can be adjusted as necessary according to factors, such as the length of the probe.

The polypeptides encoded by these novel nucleic acids are referred to herein as “Spike polypeptides” or “Spike proteins.” As used herein, these terms refer to a genus of polypeptides that further encompasses proteins having the amino acid sequence of SEQ ID NO: 4 or SEQ ID NO: 7, as well as those proteins and polypeptides having a high degree of similarity (at least 90% homology) with such amino acid sequences and which proteins and polypeptides are immunoreactive. In addition, “Spike polypeptides” and “Spike proteins” refer to those proteins encoded by nucleic acid molecules which hybridize under conditions of high stringency to the nucleic acid strand complementary to the coding sequences of SEQ ID NO: 3 or SEQ ID NO: 6.

The term “purified” as used herein, means that the Spike polypeptides are essentially free of association with other proteins or polypeptides, for example, as a purification product of recombinant host cell culture or as a purified product from a non-recombinant source. The term “substantially purified”, as used herein, refers to a mixture that contains Spike polypeptides and is essentially free of association with other proteins or polypeptides, but for the presence of known proteins that can be removed using a specific antibody, and which substantially purified Spike polypeptides can be used as antigens.

A Spike polypeptide “variant” as referred to herein means a polypeptide substantially homologous to native Spike polypeptides, but which has an amino acid sequence different from that of native Spike polypeptides because of one or more deletions, insertions, or substitutions. The variant amino acid sequence preferably is at least 80% identical to a native Spike polypeptide amino acid sequence, most preferably at least 90% identical. The percent identity can be determined, for example by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps.

Variants can comprise conservatively substituted sequences, meaning that a given amino acid residue is replaced by a residue having similar physiochemical characteristics. Examples of conservative substitutions include substitution of one aliphatic residue for another, such as IIe, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring Spike polypeptide variants are also encompassed by the invention. Examples of such variants are proteins that result from alternate mRNA splicing events or from proteolytic cleavage of the Spike polypeptides. Variations attributable to proteolysis include, for example, differences in the termini upon expression in different types of host cells, due to proteolytic removal of one or more terminal amino acids from the Spike polypeptides. Variations attributable to frameshifting include, for example, differences in the termini upon expression in different types of host cells due to different amino acids.

As stated above, the invention provides isolated and purified, or homogeneous, Spike polypeptides, both recombinant and non-recombinant. Variants and derivatives of native Spike polypeptides that can be used as antigens can be obtained by mutations of nucleotide sequences coding for native Spike polypeptides. Alterations of the native amino acid sequence can be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion, or insertion. Exemplary methods of making the alterations set forth above are disclosed by Walder et al. (Gene 42:133, 1986); Bauer et al. (Gene 37:73, 1985); Craik (BioTechniques, Jan. 12-19, 1985); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462, all of which are incorporated by reference.

Within an aspect of the invention, Spike polypeptides can be utilized to prepare antibodies that specifically bind to Spike polypeptides. The term “antibodies” is meant to include polyclonal antibodies, monoclonal antibodies, fragments thereof such as F(ab′)2 and Fab fragments, as well as any recombinantly produced binding partners. Antibodies are defined to be specifically binding if they bind Spike polypeptides with a Kₐ of greater than or equal to about 10⁷ M⁻¹. Affinities of binding partners or antibodies can be readily determined using conventional techniques, for example, those described by Scatchard et al., Ann. N.Y Acad. Sci., 51:660 (1949). Polyclonal antibodies can be readily generated from a variety of sources, for example, horses, cows, goats, sheep, dogs, chickens, rabbits, mice, or rats, using procedures that are well known in the art.

The invention further encompasses isolated fragments and oligonucleotides derived from the nucleotide sequence of SEQ ID NOS: 2-3 & 6. The invention also encompasses polypeptides encoded by these fragments and oligonucleotides.

Due to the known degeneracy of the genetic code, wherein more than one codon can encode the same amino acid, a DNA sequence can vary from that shown in SEQ ID NOS: 2-3 & 6 and still encode a Spike polypeptide having the amino acid sequence of SEQ ID NO: 7. Such variant DNA sequences can result from silent mutations (e.g., occurring during PCR amplification), or can be the product of deliberate mutagenesis of a native sequence.

The invention thus provides equivalent isolated DNA sequences, encoding Spike polypeptides, selected from: (a) nucleic acid molecules comprising SEQ ID NOS: 2-3 & 6; (b) DNA capable of hybridization to SEQ ID NOS: 3 or 6 under conditions of high stringency; (c) nucleic acid molecules comprising fragments of SEQ ID NOS: 2-3 & 6; and (d) nucleic acid molecules which are degenerate as a result of the genetic code to a DNA defined in (a), (b), or (c) and which encode Spike polypeptides and fragments of Spike polypeptides. Spike polypeptides encoded by such nucleic acid equivalent sequences are encompassed by the invention.

Examples of Spike polypeptides encoded by DNA equivalent to SEQ ID NOS: 3 or 6, include, but are not limited to, Spike polypeptide fragments and Spike polypeptides comprising inactivated N-glycosylation site(s), inactivated protease processing site(s), or conservative amino acid substitution(s), as described above.

Recombinant expression vectors containing a nucleic acid sequence encoding Spike polypeptides can be prepared using well known methods. The expression vectors include a Spike DNA sequence operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are “operably linked” when the regulatory sequence functionally relates to the Spike DNA sequence. Thus, a promoter nucleotide sequence is operably linked to a Spike DNA sequence if the promoter nucleotide sequence controls the transcription of the Spike DNA sequence. The ability to replicate in the desired host cells, usually conferred by an origin of replication, and a selection gene by which transformants are identified can additionally be incorporated into the expression vector.

In addition, sequences encoding appropriate signal peptides that are not naturally associated with Spike polypeptides can be incorporated into expression vectors.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids. Commercially available vectors include those that are specifically designed for the expression of proteins. These include pMAL-p2 and pMAL-c2 vectors, which are used for the expression of proteins fused to maltose binding protein (New England Biolabs, Beverly, Mass., USA).

Specific examples of plasmids comprising optimized Spike genes of SARS CoV include the following:

| | |
|---|---|
| pPCR-Script-040078 | deposited at C.N.C.M. on |
| | Jun. 8, 2004 under the |
| | number I-3221; |
| pcDNA-Spike-HKUPRC-040086 | deposited at C.N.C.M. on |
| | Jun. 8, 2004 under the |
| | number I-3222; and |
| pcSFV-HKUPRC-040091 | deposited at C.N.C.M. on |
| | Jun. 8, 2004 under the |
| | number I-3223. |

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776), and tac promoter (Maniatis, Molecular Cloning: A Laboratory Manual, Cold Spring Harbor Laboratory, p. 412, 1982).

Suitable host cells for expression of Spike polypeptides include prokaryotes, yeast or higher eukaryotic cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems can also be employed to produce Spike polypeptides using RNAs derived from DNA constructs disclosed herein.

It will be understood that the present invention is intended to encompass the previously described proteins in isolated or purified form, whether obtained using the techniques described herein or other methods. In a preferred embodiment of this invention, the Spike polypeptides are substantially free of human tissue and human tissue components, nucleic acids, extraneous proteins and lipids, and adventitious microorganisms, such as bacteria and viruses. It will also be understood that the invention encompasses equivalent proteins having substantially the same biological and immunogenic properties. Thus, this invention is intended to cover serotypic variants of the proteins of the invention.

Depending on the use to be made of the Spike polypeptides of the invention, it may be desirable to label them. Examples of suitable labels are radioactive labels, enzymatic labels, fluorescent labels, chemiluminescent labels, and chromophores. The methods for labeling proteins and glycoproteins of the invention do not differ in essence from those widely used for labeling immunoglobulin. The need to label may be avoided by using labeled antibody to the antigen of the invention or anti-immunoglobulin to the antibodies to the antigen as an indirect marker.

Once the Spike polypeptides of the invention have been obtained, they can be used to produce polyclonal and monoclonal antibodies reactive therewith. Thus, a protein or polypeptide of the invention can be used to immunize an animal host by techniques known in the art. Such techniques usually involve inoculation, but they may involve other modes of administration. A sufficient amount of the protein or the polypeptide is administered to create an immunogenic response in the animal host. Any host that produces antibodies to the antigen of the invention can be used. Once the animal has been immunized and sufficient time has passed for it to begin producing antibodies to the antigen, polyclonal antibodies can be recovered. The general method comprises removing blood from the animal and separating the serum from the blood. The serum, which contains antibodies to the antigen, can be used as an antiserum to the antigen. Alternatively, the antibodies can be recovered from the serum. Affinity purification is a preferred technique for recovering purified polyclonal antibodies to the antigen, from the serum.

Monoclonal antibodies to the antigens of the invention can also be prepared. One method for producing monoclonal antibodies reactive with the antigens comprises the steps of immunizing a host with the antigen; recovering antibody producing cells from the spleen of the host; fusing the antibody producing cells with myeloma cells deficient in the enzyme hypoxanthine-guanine phosphoribosyl transferase to form hybridomas; selecting at least one of the hybridomas by growth in a medium comprising hypoxanthine, aminopterin, and thymidine; identifying at least one of the hybridomas that produces an antibody to the antigen, culturing the identified hybridoma to produce antibody in a recoverable quantity; and recovering the antibodies produced by the cultured hybridoma.

These polyclonal or monoclonal antibodies can be used in a variety of applications. Among these is the neutralization of corresponding proteins. They can also be used to detect viral antigens in biological preparations or in purifying corresponding proteins, glycoproteins, or mixtures thereof, for example when used in an affinity chromatographic columns.

The Spike polypeptides can be used as antigens to identify antibodies to SARS CoV in materials and to determine the concentration of the antibodies in those materials. Thus, the antigens can be used for qualitative or quantitative determination of the virus in a material. Such materials include human tissue and human cells, as well as biological fluids, such as human body fluids, including human sera. When used as a reagent in an immunoassay for determining the presence or concentration of the antibodies to SARS CoV, the antigens of the present invention provide an assay that is convenient, rapid, sensitive, and specific.

More particularly, the antigens of the invention can be employed for the detection of SARS CoV by means of immunoassays that are well known for use in detecting or quantifying humoral components in fluids. Thus, antigen-antibody interactions can be directly observed or determined by secondary reactions, such as precipitation or agglutination. In addition, immunoelectrophoresis techniques can also be employed. For example, the classic combination of electrophoresis in agar followed by reaction with anti-serum can be utilized, as well as two-dimensional electrophoresis, rocket electrophoresis, and immunolabeling of polyacrylamide gel patterns (Western Blot or immunoblot). Other immunoassays in which the antigens of the present invention can be employed include, but are not limited to, radioimmunoassay, competitive immunoprecipitation assay, enzyme immunoassay, and immunofluorescence assay. It will be understood that turbidimetric, colorimetric, and nephelometric techniques can be employed. An immunoassay based on Western Blot technique is preferred.

Immunoassays can be carried out by immobilizing one of the immunoreagents, either an antigen of the invention or an antibody of the invention to the antigen, on a carrier surface while retaining immunoreactivity of the reagent. The reciprocal immunoreagent can be unlabeled or labeled in such a manner that immunoreactivity is also retained. These techniques are especially suitable for use in enzyme immunoassays, such as enzyme linked immunosorbent assay (ELISA) and competitive inhibition enzyme immunoassay (CIEIA).

When either the antigen of the invention or antibody to the antigen is attached to a solid support, the support is usually a glass or plastic material. Plastic materials molded in the form of plates, tubes, beads, or disks are preferred. Examples of suitable plastic materials are polystyrene and polyvinyl chloride. If the immunoreagent does not readily bind to the solid support, a carrier material can be interposed between the reagent and the support. Examples of suitable carrier materials are proteins, such as bovine serum albumin, or chemical reagents, such as gluteraldehyde or urea. Coating of the solid phase can be carried out using conventional techniques.

The invention provides immunogenic Spike polypeptides, and more particularly, protective polypeptides for use in the preparation of vaccine compositions against SARS CoV. These polypeptides can thus be employed as viral vaccines by administering the polypeptides to a mammal susceptible to SARS CoV infection. Conventional modes of administration can be employed. For example, administration can be carried out by oral, respiratory, or parenteral routes. Intradermal, subcutaneous, and intramuscular routes of administration are preferred when the vaccine is administered parenterally.

Various methods for achieving adjuvant effect for the vaccine include the use of agents, such as aluminum hydroxide or phosphate (alum), commonly used as 0.05 to 0.1 percent solution in phosphate buffered saline, admixture with synthetic polymers of sugars (Carbopol) used as 0.25% solution. Another suitable adjuvant compound comprises DDA (dimethyldioctadecyl-ammonium bromide), as well as immune modulating substances, such as lymphokines (e.g., IFN-gamma, IL-1, IL-2, and IL-12) or IFN-gamma inducer compounds, such as poly I:C.

The vaccine composition according to the present invention is advantageously prepared as an injectable form (either as liquid solution or suspension). However, solid forms suitable for solution in or suspension in, liquid prior injection may also be prepared.

In addition, if desired, the vaccine composition can contain minor amounts of auxiliary substances such as wetting or emulsifying agents, pH buffering agents, or adjuvants, which enhance the effectiveness of the vaccine.

The vaccine compositions of the invention are administered in a manner compatible with the dosage formulation, and in such amount as will be therapeutically effective and immunogenic. The quantity to be administered depends on the subject to be treated including, e.g., the capacity of the individual's immune system to induce an immune response.

The dosage of the vaccine will depend on the route of administration and will vary according to the age of the patient to be vaccinated and, to a lesser degree, the size of the person to be vaccinated.

The major purpose of the immune response in a SARS CoV infected mammal is to inactivate the free SARS CoV and to eliminate SARS CoV infected cells that have the potential to release infectious virus. The B-cell arm of the immune response has the major responsibility for inactivating free SARS CoV virus. The principal manner in which this is achieved is by neutralization of infectivity. Another major mechanism for destruction of the SARS CoV infected cells is provided by cytotoxic T lymphocytes (CTL) that recognize viral Spike antigens expressed in combination with class I histocompatibility antigens at the cell surface. The CTLs recognize Spike polypeptides processed within cells from a Spike protein that is produced, for example, by the infected cell or that is internalized by a phagocytic cell. Thus, this invention can be employed to stimulate a B-cell response to Spike polypeptides, as well as immunity mediated by a CTL response following viral infection. The CTL response can play an important role in mediating recovery from primary SARS CoV infection and in accelerating recovery during subsequent infections.

The ability of the Spike polypeptides and vaccines of the invention to induce protective levels of neutralizing antibody in a host can be enhanced by emulsification with an adjuvant, incorporating in a liposome, coupling to a suitable carrier, or by combinations of these techniques. For example, the Spike polypeptides of the invention can be administered with a conventional adjuvant, such as aluminum phosphate and aluminum hydroxide gel, in an amount sufficient to potentiate humoral or cell-mediated immune response in the host. Similarly, the Spike polypeptides can be bound to lipid membranes or incorporated in lipid membranes to form liposomes. The use of nonpyrogenic lipids free of nucleic acids and other extraneous matter can be employed for this purpose.

The immunization schedule will depend upon several factors, such as the susceptibility of the host to infection and the age of the host. A single dose of the vaccine of the invention can be administered to the host or a primary course of immunization can be followed in which several doses at intervals of time are administered. Subsequent doses used as boosters can be administered as needed following the primary course.

The Spike proteins, polypeptides, and vaccines of the invention can be administered to the host in an amount sufficient to prevent or inhibit SARS CoV infection or replication in vivo. In any event, the amount administered should be at least sufficient to protect the host against substantial immunosuppression, even though SARS CoV infection may not be entirely prevented. An immunogenic response can be obtained by administering the Spike proteins or glycoproteins of the invention to the host in an amount of about 10 to about 500 micrograms antigen per kilogram of body weight, preferably about 50 to about 100 micrograms antigen per kilogram body weight. The proteins and vaccines of the invention can be administered together with a physiologically acceptable carrier. For example, a diluent, such as water or a saline solution, can be employed.

Another aspect of the invention provides a method of RNA and/or DNA vaccination. The method also includes administering any combination of the nucleic acids encoding Spike polypeptides, the proteins and polypeptides per se, with or without carrier molecules, to an individual. In embodiments, the individual is an animal, and is preferably a mammal. More preferably, the mammal is selected from the group consisting of a human, a mouse, a rat, a rabbit, a sheep, a dog, a cat, a bovine, a pig, and a horse. In an especially preferred embodiment, the mammal is a human.

The methods of treating include administering immunogenic compositions comprising Spike polypeptides, but compositions comprising nucleic acids encoding Spike polypeptides as well. Those of skill in the art are cognizant of the concept, application, and effectiveness of nucleic acid vaccines and nucleic acid vaccine technology as well as protein and polypeptide based technologies. The nucleic acid based technology allows the administration of nucleic acids encoding Spike polypeptides, naked or encapsulated, directly to tissues and cells without the need for production of encoded proteins prior to administration. The technology is based on the ability of these nucleic acids to be taken up by cells of the recipient organism and expressed to produce an immunogenic determinant to which the recipient's immune system responds. Typically, the expressed antigens are displayed on the surface of cells that have taken up and expressed the nucleic acids, but expression and export of the encoded antigens into the circulatory system of the recipient individual is also within the scope of the present invention. Such nucleic acid vaccine technology includes, but is not limited to, delivery of naked DNA and RNA and delivery of expression vectors encoding Spike polypeptides. Although the technology is termed “vaccine”, it is equally applicable to immunogenic compositions that do not result in a protective response. Such non-protection inducing compositions and methods are encompassed within the present invention.

Although it is within the present invention to deliver nucleic acids encoding Spike polypeptides and carrier molecules as naked nucleic acid, the present invention also encompasses delivery of nucleic acids as part of larger or more complex compositions. Included among these delivery systems are viruses, virus-like particles, or bacteria containing the nucleic acid encoding Spike polypeptides. Also, complexes of the invention's nucleic acids and carrier molecules with cell permeabilizing compounds, such as liposomes, are included within the scope of the invention. Other compounds, such as molecular vectors (EP 696,191, Samain et al.) and delivery systems for nucleic acid vaccines are known to the skilled artisan and exemplified in, for example, WO 93 06223 and WO 90 11092, U.S. Pat. No. 5,580,859, and U.S. Pat. No. 5,589,466 (Vical's patents), which are incorporated by reference herein, and can be made and used without undue or excessive experimentation.

Protein based SARS vaccine can induce a neutralizing and protective antibody-dependent immune response after a single or double injection of Spike protein. Protein based vaccines present considerable safety advantages over vector-expressed (i.e., plasmid, MVA, Adeno) or whole inactivated virus vaccine.

To further achieve the objects and in accordance with the purposes of the present invention, a kit capable of diagnosing a SARS CoV infection is described. This kit, in one embodiment, contains the antibodies of this invention, which are capable of binding to SARS CoV Spike polypeptide. This kit, in another embodiment, contains the polypeptides of this invention, which are capable of detecting the presence or absence of antibodies, which bind to the Spike polypeptide. This kit, in yet another embodiment, contains the nucleic acid molecules of this invention, which are capable of hybridizing to viral RNA or analogous DNA sequences to indicate the presence of a SARS CoV infection. Different diagnostic techniques can be used which include, but are not limited to: (I) Southern blot procedures to identify cellular DNA which may or may not be digested with restriction enzymes; (2) Northern blot techniques to identify RNA extracted from cells; and (3) dot blot techniques, i.e., direct filtration of the sample through an ad hoc membrane, such as nitrocellulose or nylon, without previous separation on agarose gel; (4) immunoassay based on Western blot technique; (5) ELISA (enzyme linked immunosorbent assay); (6) FACS; (7) indirect immunofluorescent assay; or (8) immunoprecipitation assay. Suitable material for dot blot technique could be obtained from body fluids including, but not limited to, serum and plasma, supernatants from culture cells, or cytoplasmic extracts obtained after cell lysis and removal of membranes and nuclei of the cells by centrifugation.

This invention will be described in greater detail in the following Examples.

### EXAMPLE 1

The Spike gene from a patient infected with the SARS CoV, designated as Spike-Pasteur (SEQ ID NO: 1), was obtained. Spike-Pasteur was cloned into a pcDNA eukaryotic expression vector and transfected into 293T cells. Cells transfected with pcDNA-Spike-Pasteur did not express Spike-Pasteur polypeptide, as no detectable levels of Spike protein were seen either by FACS or by Western blot.

Spike-Pasteur was subsequently expressed in the SFV viral expression vector, which effectively allowed for expression in transfected BHK cells. However, the yield of SFV viral particles was:low, because of the presence of 2 Spe I sites in the Spike gene. Spe I is usually used to linearize the plasmid at the end of the SFV coding sequence. Because Spe I could not be used, PSFV-Spike-Pasteur was linearized with Sph I, resulting in additional 3′ RNA sequences of >2000 bases of vector RNA. The level of protein expression from SFV-Spike-Pasteur infected cells was weak.

In an effort to improve the expression of Spike-Pasteur, two Spe I sites in the Spike-Pasteur sequence were eliminated, resulting in Spike-Pasteur-modif (SEQ ID NO: 2). Spike-Pasteur-modif allowed for standard linearization of pSFV with Spe I, and was found to increase the yield of SFV particle production up to 100-fold. RNA transfection from Sph I linearized pSFV-Spike-Pasteur usually yields SFV titer of 2×10⁷ IP/m. RNA transfection from Spe I linearized pSFV-Spike-Pasteur-modif usually yields SFV titer of 1-2×10⁹ IP/ml.

### EXAMPLE 2

The Spike-Pasteur sequence was further subjected to a bioinformatics analysis. The cDNA for Spike-Pasteur contains numerous cis-acting sites which may negatively influence expression. To further improve expression, 32 of the identified 33 negative cis-acting signals were eliminated from Spike-Pasteur, and additional signals to stimulate gene expression were added, producing Spike-HKU-PRC (SEQ ID NO: 3). Spike-HKU-PRC was cloned into pSC, pcDNA, and pSFV vectors.

As shown in Table 1, 19 of 19 AU-rich RNA instability motifs present in Spike-Pasteur were eliminated in producing Spike-HKU-PRC. In addition, 11 of 12 putative splice donor and acceptor sites were removed, as well as an internal poly(A) site and a repeat sequence and secondary stretch.

TABLE 1
Negative cis-acting signals in Spike-Pasteur
compared to optimized Spike HKU-PRC.

| | Spike-Pasteur | Spike-HKU-PRC |
|---|---|---|
| AU-rich RNA instability motifs | 19 | 0 |
| Repeat sequences & Secondary | 1 | 0 |
| stretches | | |
| Splice donor and acceptor sites | 12 | 1 |
| Internal poly(A) sites | 1 | 0 |

Additional expression enhancing sequences added to Spike-HKU-PRC included a Kozak consensus sequence introduced upstream of the starting ATG to increase translation initiation, and two stop codons added to ensure efficient termination. In an effort to increase mRNA half-life, the GC-content of Spike-HKU-PRC was increased from 38% to 49%, while avoiding regions of very high (>80%) or very low (<30%) GC content. In addition, codon usage was adapted to the bias of Cricetulus griseus to increase translation efficiency. Table 2 shows the codon usage of Cricetulus griseus, with the frequency of each codon given as number per thousand codons.

The following sequences were avoided in Spike-HKU-PRC: internal TATA-boxes, chi-sites, and ribosomal entry sites; AT-rich or GC-rich sequence stretches; repeat sequences and RNA secondary structures; and splice donor and acceptor sites as well as splice branch points.

TABLE 2

The codon usage of Cricetulus griseus, as found
at http://www.kazusa.or.jp/codon/). The
frequencies are given as number per thousand.

| | | | | | | | |
|---|---|---|---|---|---|---|---|
| UUU | 19.3 | UCU | 16.1 | UAU | 12.8 | UGU | 8.9 |
| UUC | 22.0 | UCC | 16.5 | UAC | 16.3 | UGC | 10.3 |
| UUA | 6.1 | UCA | 10.1 | UAA | 0.6 | UGA | 1.1 |
| UUG | 14.1 | UCG | 3.5 | UAG | 0.6 | UGG | 13.3 |
| CUU | 12.9 | CCU | 17.3 | CAU | 10.1 | CGU | 5.8 |
| CUC | 18.1 | CCC | 17.4 | CAC | 13.0 | CGC | 9.2 |
| CUA | 7.5 | CCA | 15.5 | CAA | 10.2 | CGA | 7.1 |
| CUG | 38.6 | CCG | 4.3 | CAG | 33.8 | CGG | 10.2 |
| AUU | 17.4 | ACU | 14.2 | AAU | 17.3 | AGU | 11.6 |
| AUC | 25.0 | ACC | 20.5 | AAC | 21.1 | AGC | 16.3 |
| AUA | 6.8 | ACA | 15.7 | AAA | 24.3 | AGA | 9.9 |
| AUG | 22.8 | ACG | 4.3 | AAG | 38.4 | AGG | 10.2 |
| GUU | 11.6 | GCU | 22.7 | GAU | 24.7 | GGU | 13.2 |
| GUC | 15.9 | GCC | 25.8 | GAC | 27.9 | GGC | 21.8 |
| GUA | 7.9 | GCA | 16.5 | GAA | 27.6 | GGA | 16.3 |
| GUG | 30.2 | GCG | 4.8 | GAG | 40.9 | GGG | 13.7 |

### EXAMPLE 3

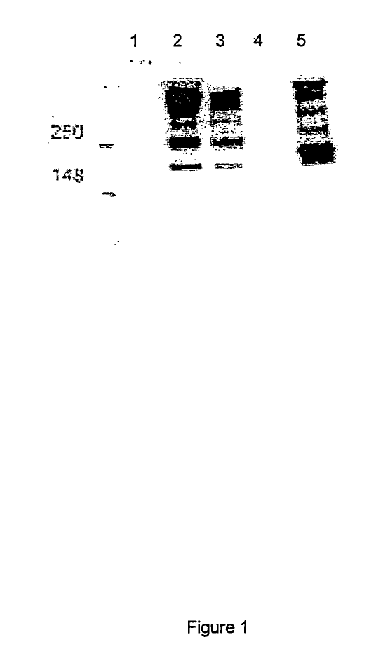

Spike protein expression of pcDNA-Spike-Pasteur was compared to pcDNA-Spike-HKU-PRC by transfection of plasmids into 293T cells using the calcium-phosphate method. No Spike protein was observed in pcDNA-Spike-Pasteur transfected cells. In contrast, high levels of Spike protein was detected in pcDNA-Spike-HKU-PRC transfected 293T cells. (FIG. 1.) The migration and oligomerization pattern of the Spike protein is consistent with previously obtained results, revealing that this plasmid allows for expression of a full length, natively conformed SARS CoV protein. These results prove that codon optimization of the Spike coding sequence dramatically improves expression results.

### EXAMPLE 4

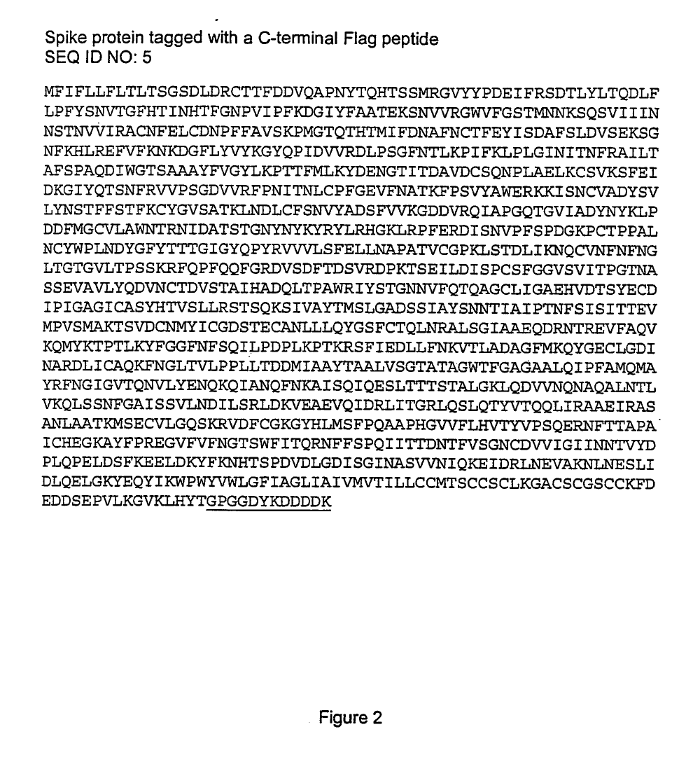

The Spike protein was tagged with a C-terminal Flag peptide, as shown in FIG. 2. The Spike protein was expressed as a full-length protein, including the C-terminal and transmembrane domains as well as a C-terminal Flag tag, in the SFV vector system previously described (See Staropoli et al., Lozach et al., and Chanel et al., all of which are herein incorporated by reference.)

Spike protein was produced alternatively from cells transfected with SFV-Spike-RNA or cells infected with SFV particles coding for SFV-Spike-RNA. To prepare SFV expression vector RNA

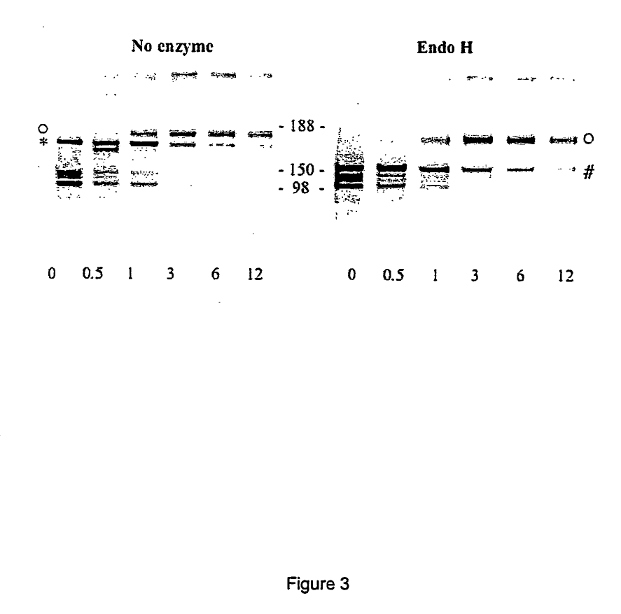

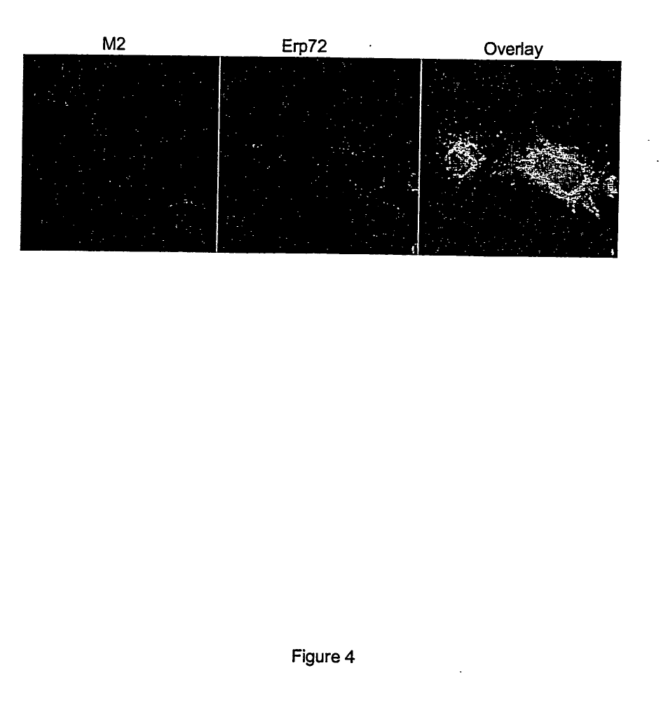

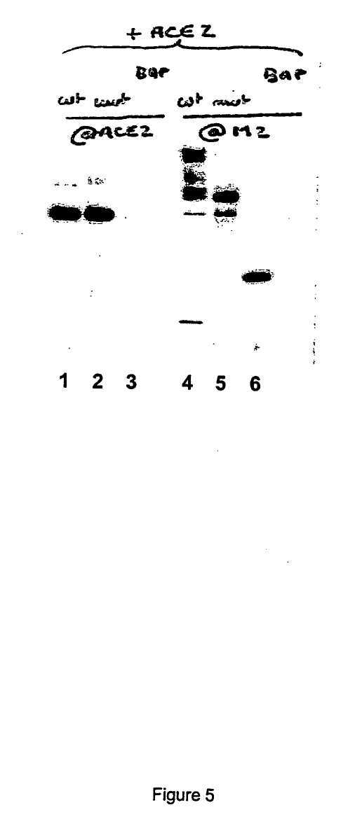

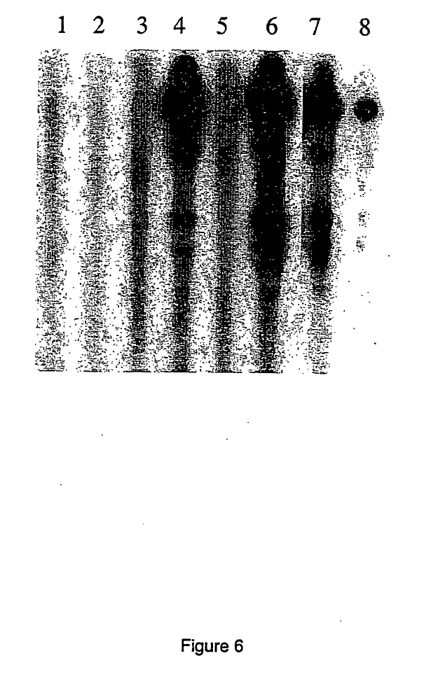

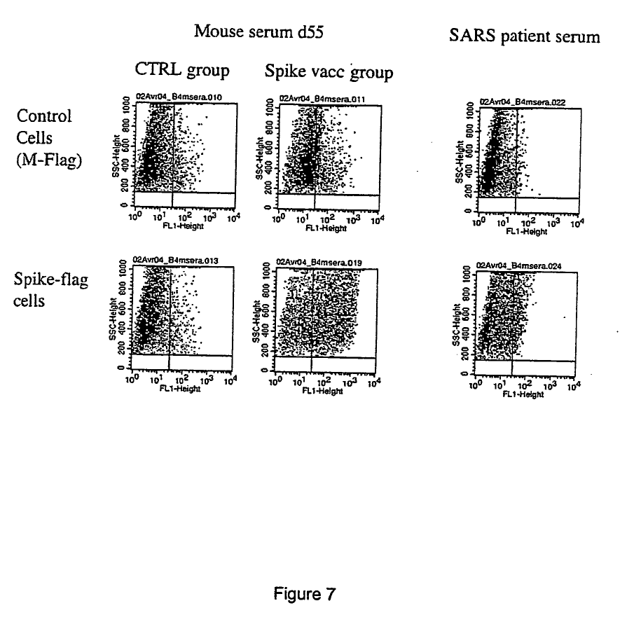

The correct folding and expected properties of the Spike protein were analyzed in a series of biochemical and immunocytochemical analyses. The protein is glycosylated after entry into the endoplasmic reticulum (ER), acquiring high mannose EndoH sensitive N-glycans (FIG. 3). The Spike protein is correctly folded, i.e. in its native conformation, as evidenced by ER quality control exit, plasma membrane expression (FIG. 4), soluble ACE2 receptor binding (FIG. 5), and recognition by a SARS patient serum in a Western blot and by FACS analysis (FIGS. 6-7).

### EXAMPLE5

For RNA immunization, RNA was transcribed in vitro according to a standard published procedure.

The Spike protein was produced in BHK cells and purified under native conditions by immunoaffinity using the anti-FLAG M2 antibody. M2-bound Spike protein was eluted under native conditions with Flag peptide. Peptide and residual detergent were eliminated by dialysis.

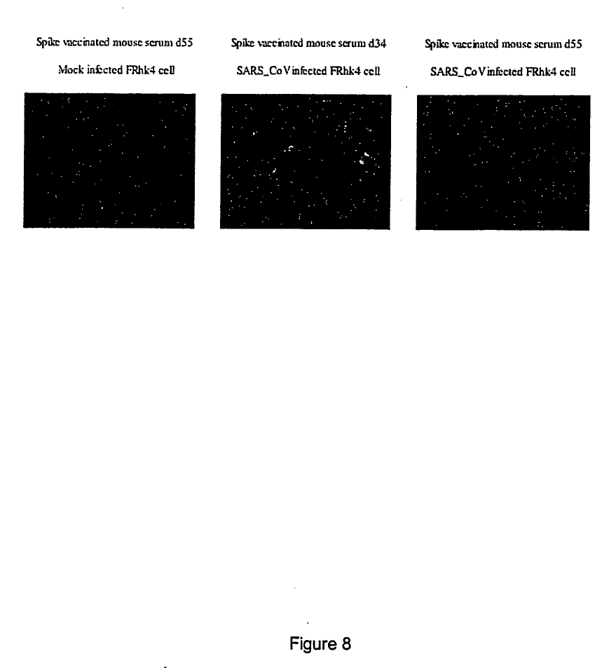

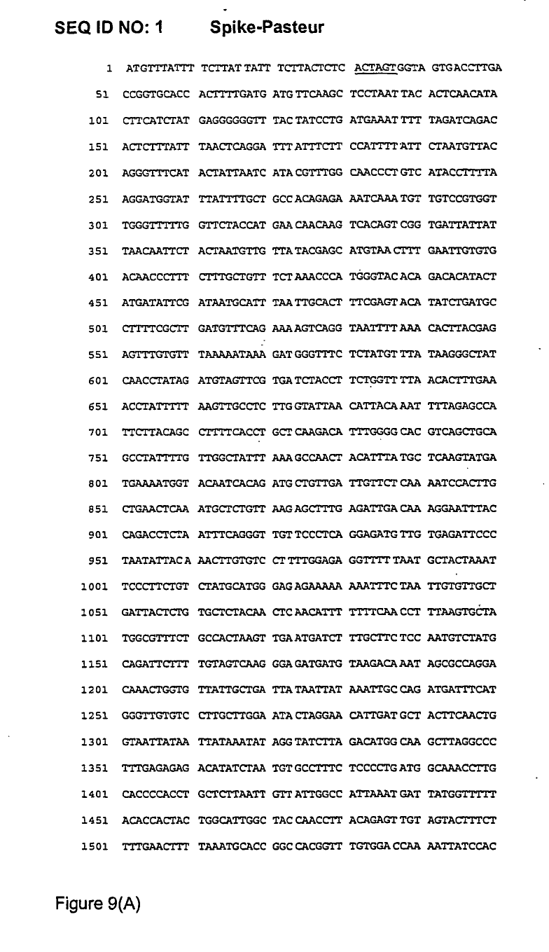

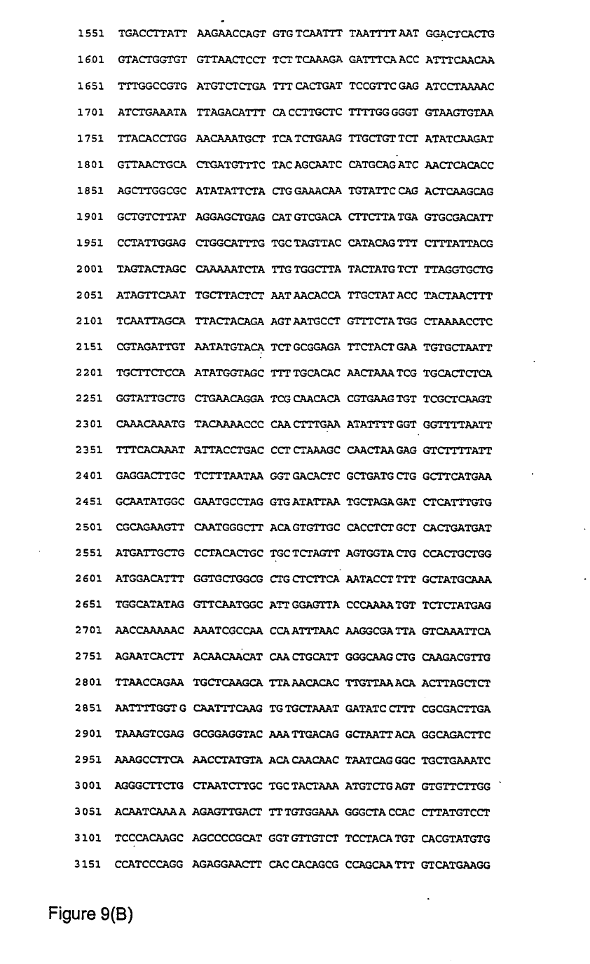

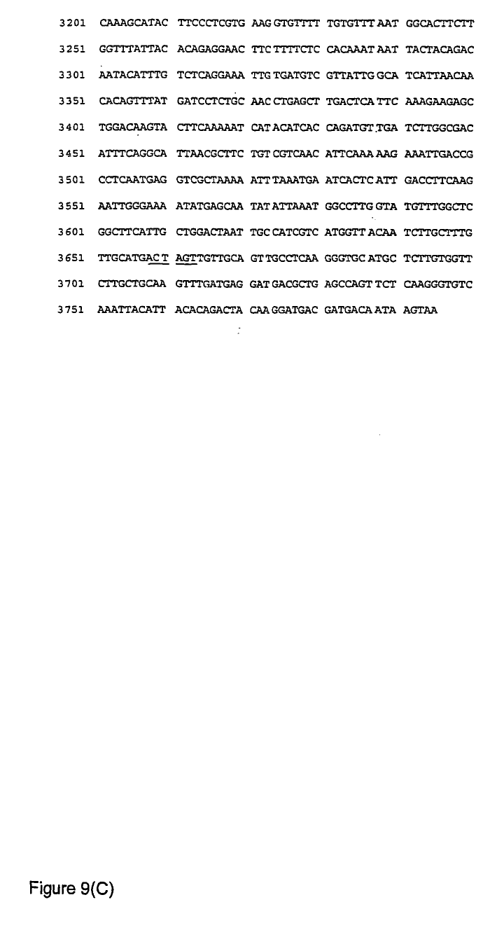

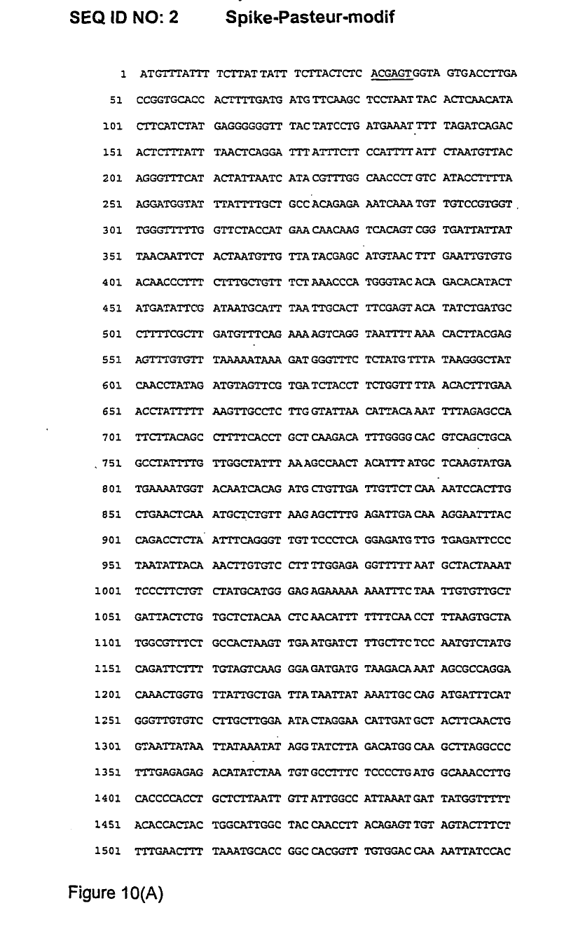

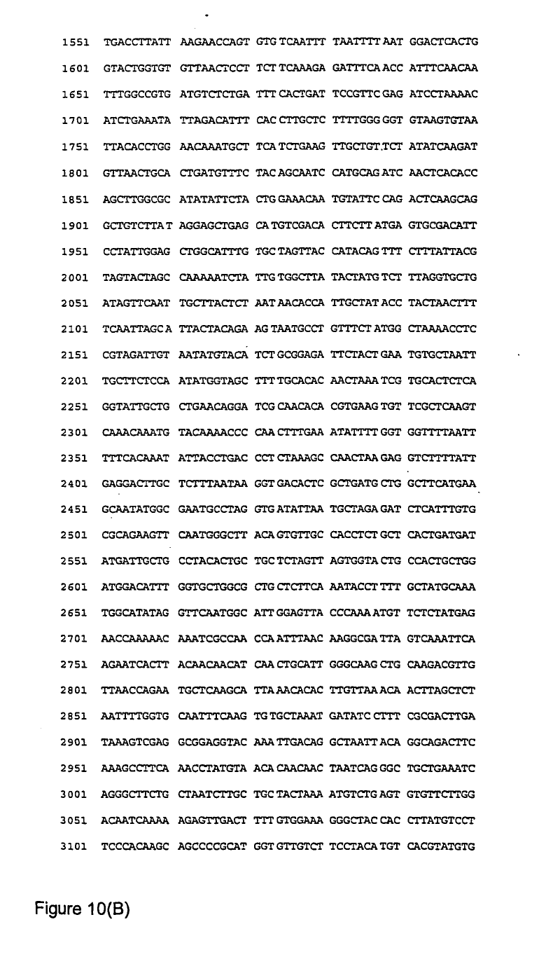

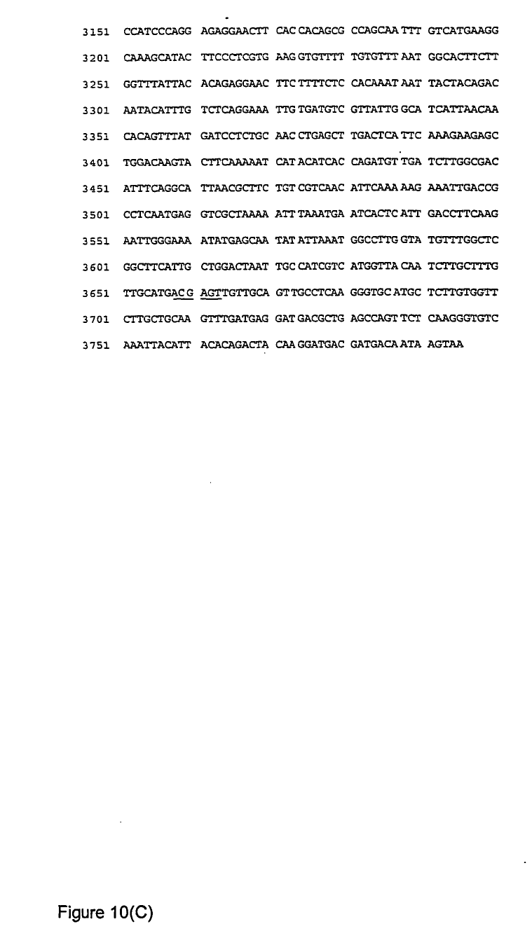

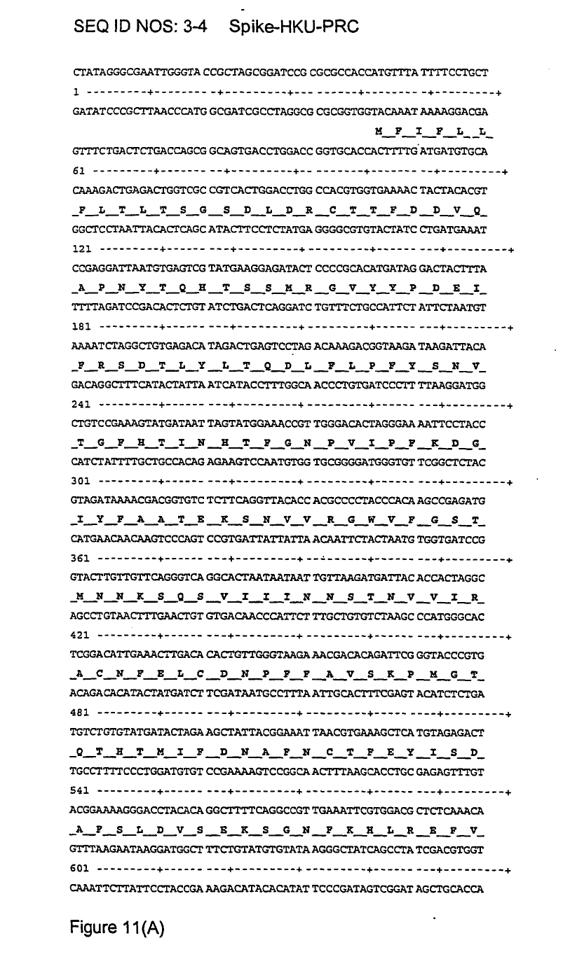

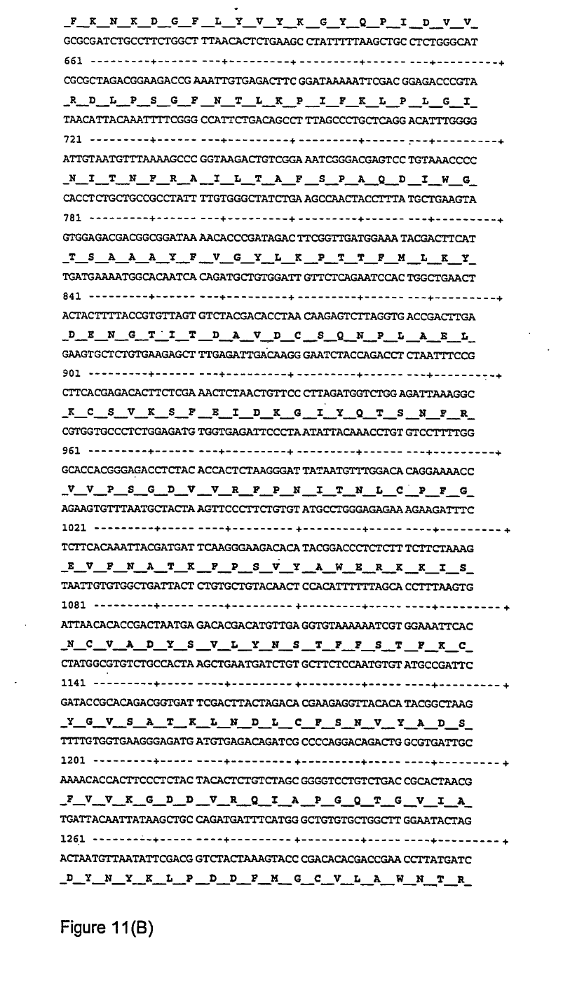

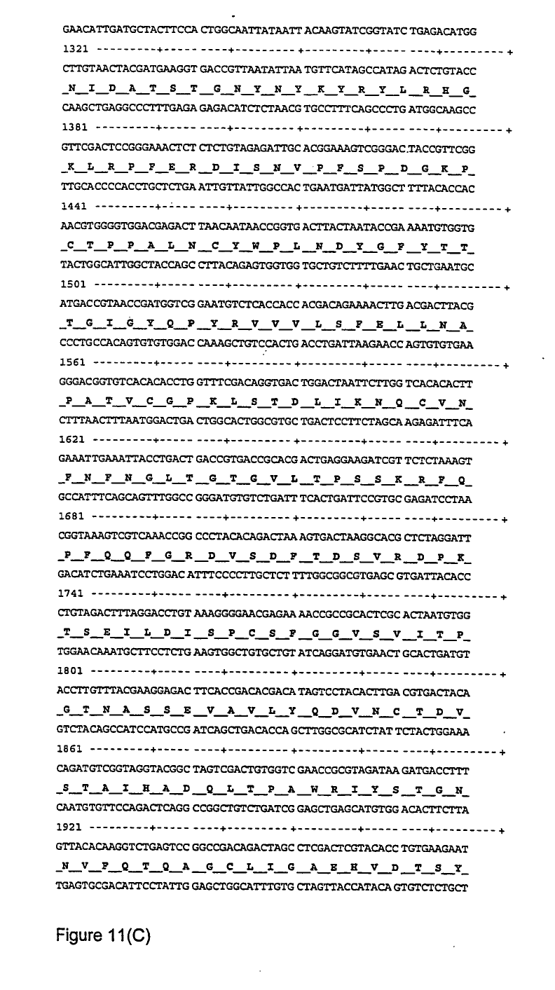

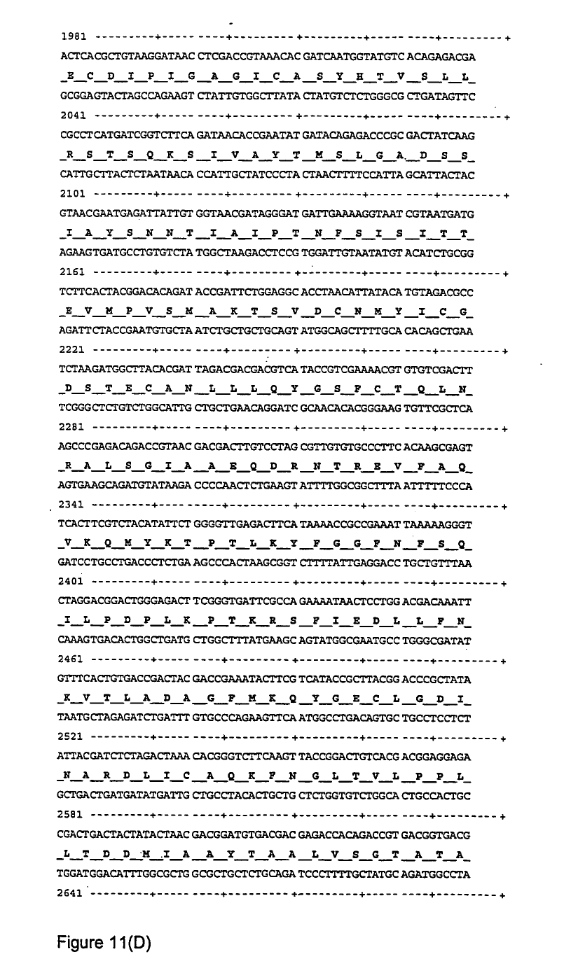

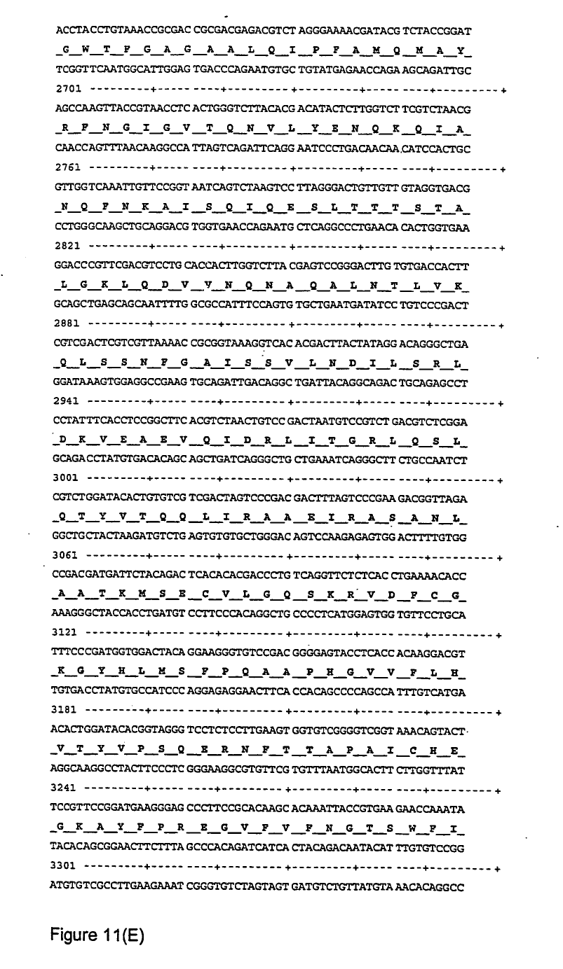

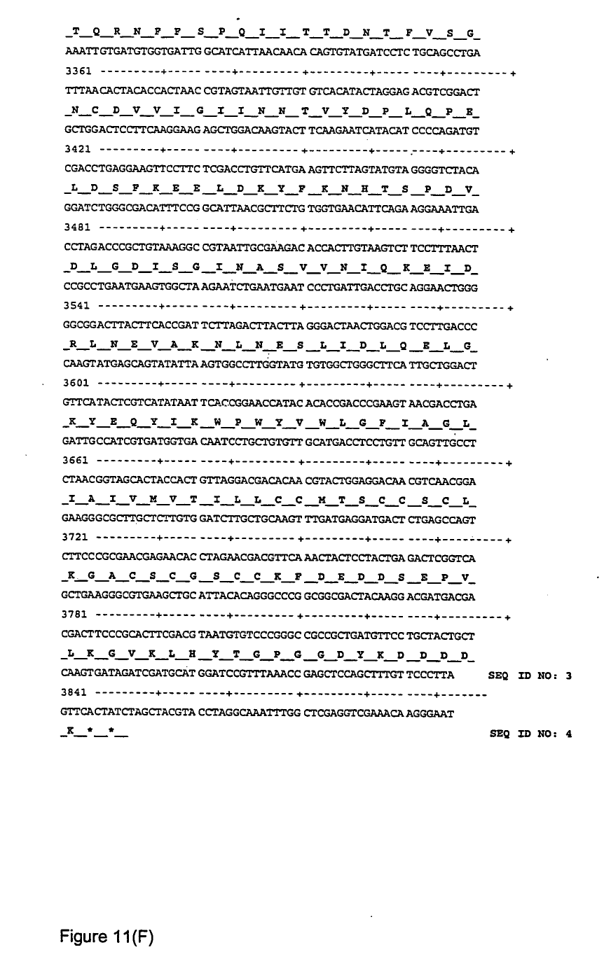

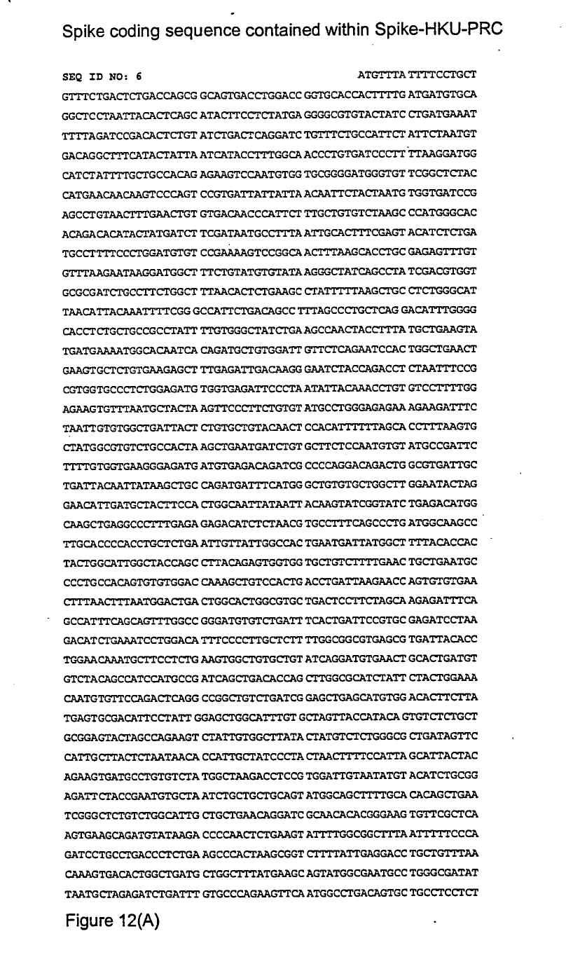

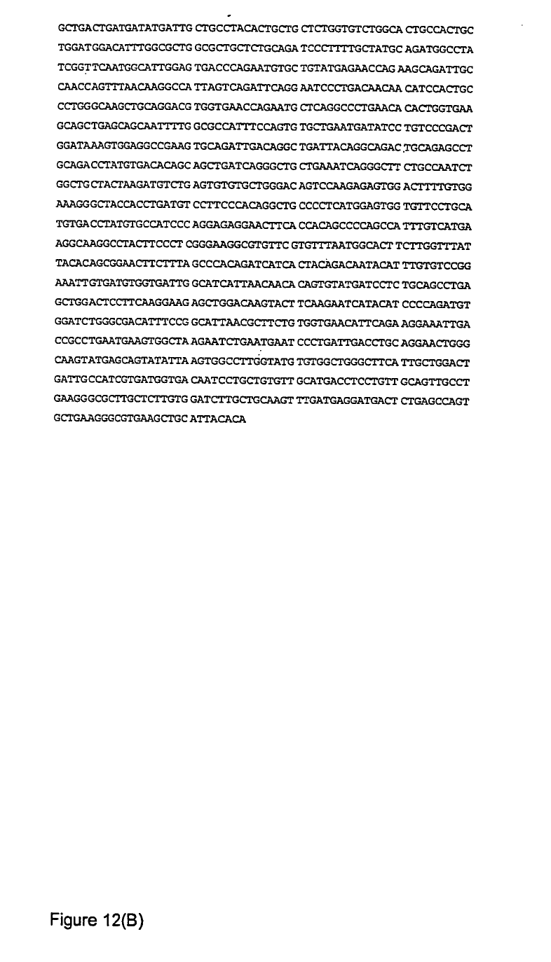

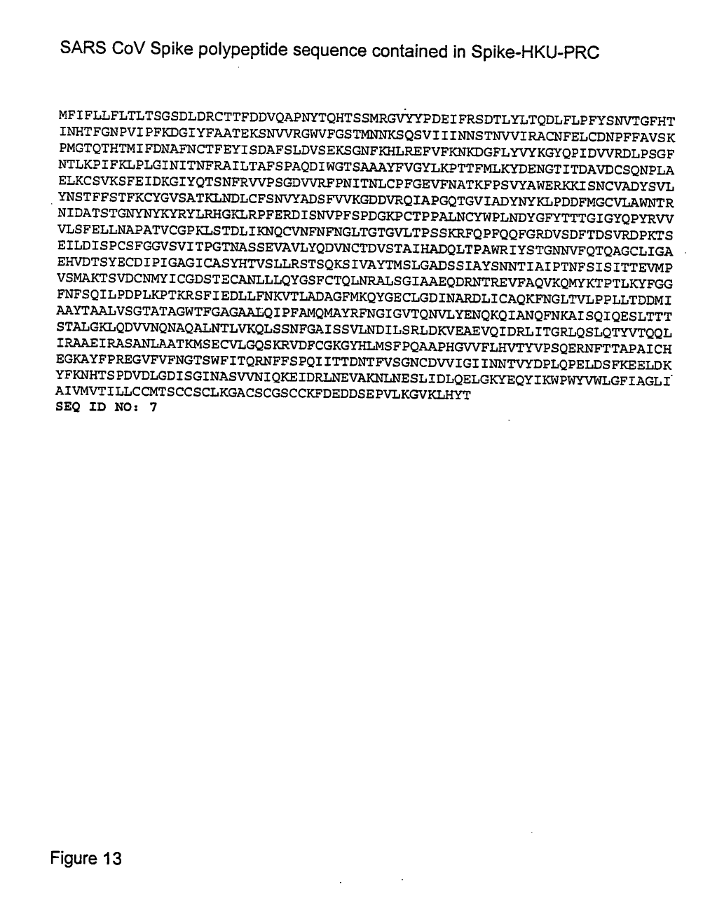

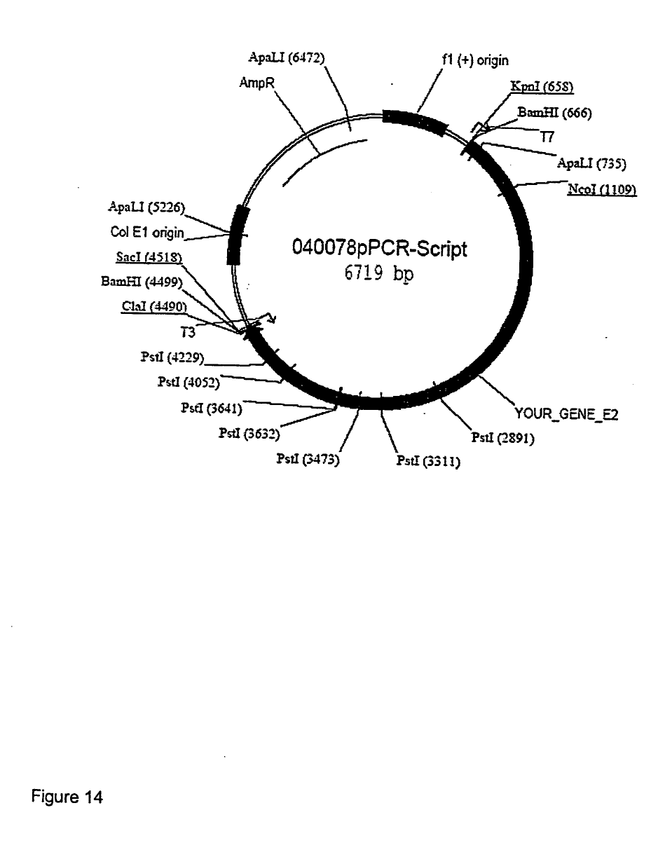

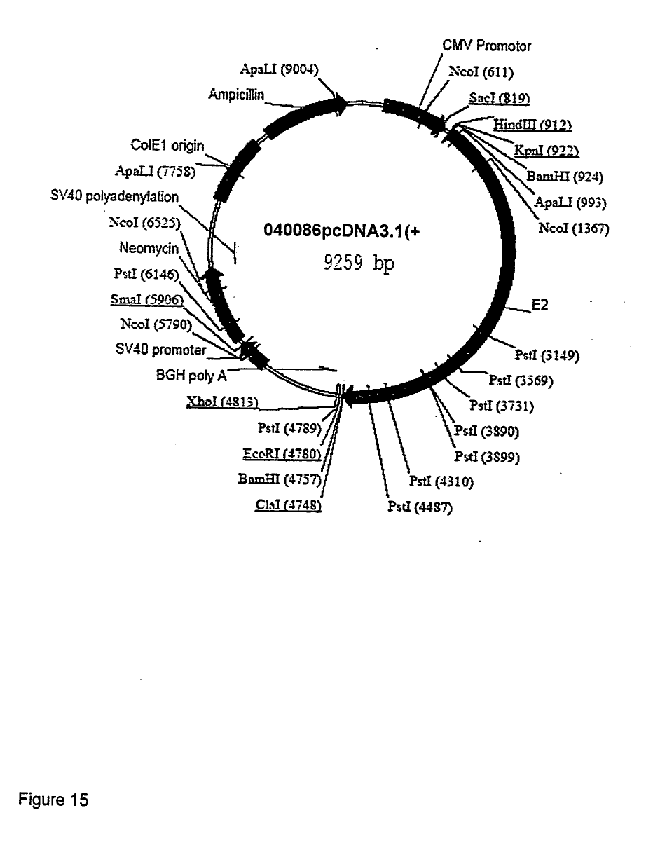

Mice were immunized intramuscularly with SFV Spike RNA, followed by intraperitoneal (IP) injection of Spike protein at day 14 and at day 35. Serum taken at day 34, day 42, and day 55 from immunized mice showed the presence of recombinant Spike-specific antibodies by Western Blot (FIG. 6), FACS (FIG. 7), and SARS CoV-specific antibodies by immunofluorescence on SARS CoV infected FRHK4 cells (FIG. 8.) These data indicate that the Spike protein expressed in the SFV vector could be successfully immunopurified in its native conformation, and that this purified protein induces high titer anti-SARS antibodies in mice.

### EXAMPLE 6

The following reagents and methods can be used in practicing this invention.

Production of SARS CoV Spike Subunit Vaccine

1/Preparation of SFV Expression Vector RNA

Note: Spike protein can be produced, for example, from cells transfected with SFV-Spike-RNA or cells infected with SFV particles coding for SFV-Spike-RNA. Here an electroporaton procedure is detailed.

Prepare 1.2×10⁷ cell/ml suspension for electroporation under STERILE conditions
1. Preparation of medium without Serum, mix the following ingredients and filter/sterilize it:
   i. Hepes 5% 10 mL
   ii. Tryptose-phosphate broth 50 mL
   iii. Penicillin 100 U/mL, Streptomicin 100 μg/mL:5mL
   iv. GMEM QSP 500 mL
2. Preheat reagents (GMEM, trypsin, PBS with no Ca²⁺ or Mg²⁺) to 37° C.
3. Gently pipette out the old medium, do not touch the wall.
4. Rinse the cells once with 10 ml PBS, discard the wash.
5. Add 3 ml trypsin and leave the flat-bottom flasks in the hood for 4-5 min.
6. Add 17 ml fresh complete medium (GMEM 5% FCS) and resuspend cells, and transfer to 50 ml tube.
7. Do a cell count; get an equivalent of 10⁷ cells and centrifuge at 1500 rpm for 5 min.
8. Resuspend the cells in 1 ml PBS (resulting in 10⁷ cells/ml)
9. Place the suspension on ice.

2/Transfection of SFV Expression Vector RNA

Electroporation should be done for both sample and untransfected control cells.
1. Prepare two 75 ml flasks with one containing 20 ml GMEM. Label properly.
2. Using sterile P1000 filter tips, transfer 800 μl of cell suspension (in PBS with no Ca⁺² or Mg⁺²) into tube containing RNA, mix twice with pipette up & down.
3. Rapidly transfer the mixture into electroporation cuvette already placed into the electroporation chamber.
4. With the electroporator set at 830 volts, 25 μFd, and resistance set to infinity, apply two pulses, with a 2-3 second delay between each pulse. (Note: to achieve the appropriate time constant, keep the electroporation chamber covered while applying the pulse.)
5. Note the electroporation time (should be within 0.4 ms)
6. Transfer the cells into the 75 ml flask containing 20 ml GMEM. Gently pipette up and down to resuspend the cells.
7. Do the same procedure for the control untransfected cells
8. Incubate the cells overnight (around 16 hours) at 37° C., 5% CO₂.

3/Cell Lysis and Protein Preparation

Preparation of cell lysate for Western blot and immunopurification

Lysis Buffer

| Triton X-100 | 1% | |
|---|---|---|
| Tris-HCl, pH 7.5 | 20 | mM |
| NaCl | 150 | mM |
| EDTA | 1 | mM |
| PMSF | 50 | mg/ml |

1×PBS without Ca+², Mg+²
Protein sample buffer without DTT
DTT, 1 M, −20° C.
Cell scraper

Prepare fresh 20-ml Lysis buffer;
1. Remove medium from flask;
2. Wash cells with 10 ml 1×PBS (one flask);
3. Add 500 μl Lysis buffer, remove cells from flask with aid of cell scraper;
4. Carefully transfer cell lysate to 1.5 ml eppendorf;
5. Remove as much residual cells with additional 300 μl Lysis buffer;
6. Keep the tube on ice for 15 min;
7. Centrifuge tube for 15 min at 13,000 rpm at 4° C. for removal of nuclear materials;
8. Transfer clear supernatant to fresh tube on ice;
9. Keep lysate extract on ice.

4/Immunoaffinity Purification of S-Flag Protein from Cell Lysates

Triton×100 Lysis Buffer (Triton×100 1%, Tris HCl pH7.5 20 mM, NaCl 150 mM, EGTA 1 mM, PMSF 50 μg/ml)

1. Take 100 μl of Anti-flag M2 agarose beads into a 1.5 ml eppendorf for each cell lysate sample using a wide boring 200 μl pipette tip.
2. Equilibrate each tube of beads with 1 ml lysis buffer. Wash 3 times (spin down the beads at full speed of centrifuge for 15 seconds, gently pipette out ˜90% PBS, avoid sucking up the beads).
3. Reserve 50 μl of cell lysate for later use.
4. Incubate the washed beads with the rest of the cell lysates by thoroughly mixing up (gentle rotation) the bead-lysate mixture at 4° C. for 4 hours.
5. Spin down the beads and remove the supernatant from each sample tube.
6. Wash the beads with 0.5 ml 1× washing buffer 3 times (spin down, add new washing buffer).
7. Spin down the beads and take out most of the supernatant so that the residue volume is about 100 μl in each sample tube,
8. Distribute 20 μl of each bead sample into a new 1.5 ml eppendorf for western blot detection.
9. The remaining bead sample tubes are stored at −20° C. for later elution.
   Elution of Protein from Immunoprecipitation
1. Procedure is according to the FLAGIPT-1 instruction manual.
2. elution with 3× FLAG peptide.
3. Prepare working 3× FLAG peptide by adding 3 ul of 5 ug/ul 3× FLAG peptide with 100 ul of 1× wash buffer.
4. Add 100 ul working 3× FLAG elution solution to the resin.
5. Incubate the mixture for 1 h at 4° C. with gentle rotation.
6. Centrifuge the resin for 10 seconds at 13000 rpm.
7. Keep the supernatant and repeat steps 4-6 three more times.
   Concentration and Purification of Protein by Amicon Filter Unit
1. After incubation collect the supernatant of each flask in 50 mL tube.
2. Centrifuge at 2000 rpm 5 minutes to remove cell pellet.
3. Transfer the 15 mL of supernatant in another 50 mL tube (with 57 μL of 100 mM solution in iPrOH of PMSF) OPTIONAL
4. Put in ice.
5. Add up to 20 ml of sample to the Amicon Ultra-15 filter unit.
6. Place capped filter device into the centrifuge rotor, with the volume graduation facing up, counterbalance with a similar device.
7. Spin at maximum 4000×g for 20 min in a swinging bucket rotor.
8. Recover the concentrated solute (500 ul) by pipetting the sample from the filter unit.

### EXAMPLE 7

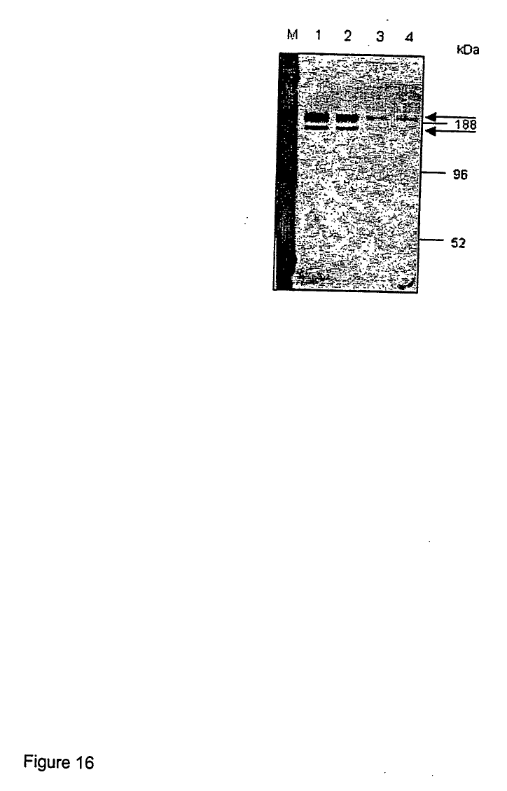

The candidate vaccine preparation, trimeric S-protein (TriSpike, the same protein described as Spike-HKU-PRC), was demonstrated to be >90% pure. A sample of TriSpike purified for vaccination studies of mice and hamsters was denatured in SDS/DTT buffer (50 mM DDT) to dissociate the trimeric protein completely into monomers. After separation by 4-12% SDS-PAGE, the gel was subjected to Silver stain (Current Protocols in Immunology Chapter 8, 9.1-9.10)) to reveal all of the proteins contained in the sample. FIG. 16 shows that only monomeric S-protein can be detected in its complex glycosylated and high-mannose forms. The degree of purity is >90%.

### EXAMPLE 8

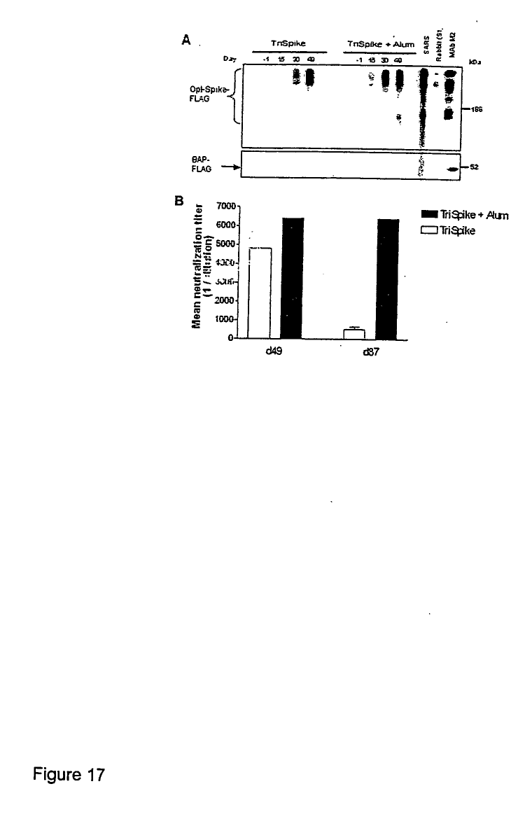

An enhanced serum IgG response was obtained in animals immunized with TriSpike in Alum adjuvant. Previous studies on the mucosal and systemic response to recombinant HagB from Porphyromonas gingivalis indicated that a higher serum IgG and mucosal IgA response from HagB+alum was induced compared to HagB without adjuvant immunization in Balb/c mice (Vaccine, 2003, 21, 4459-4471). TriSpike candidate vaccine was analyzed to determine whether it could induce not only serum IgG, but also mucosal IgA with neutralizing ability for SARS CoV. TriSpike preparation in PBS was compared with TriSpike preparation in Alum adjuvant for their capacity to induce SARS CoV specific serum IgG. Two groups of mice were immunized by the intraperitoneal route: group A represents mice which received 3 doses of 20 μg of TriSpike protein alone and group B represents mice which received 3 doses of 20 μg of TriSpike pre-mixed with 1 mg of Alum adjuvant. Western blot analysis indicated a stronger antibody response from mice immunized with TriSpike+alum as compared with mice immunized with TriSpike alone (FIG. 17). The TriSpike+Alum group also showed a higher neutralization titer (FIG. 17). TriSpike+Alum adjuvant induced a strong neutralizing and long lasting serum IgG response.

### EXAMPLE 9

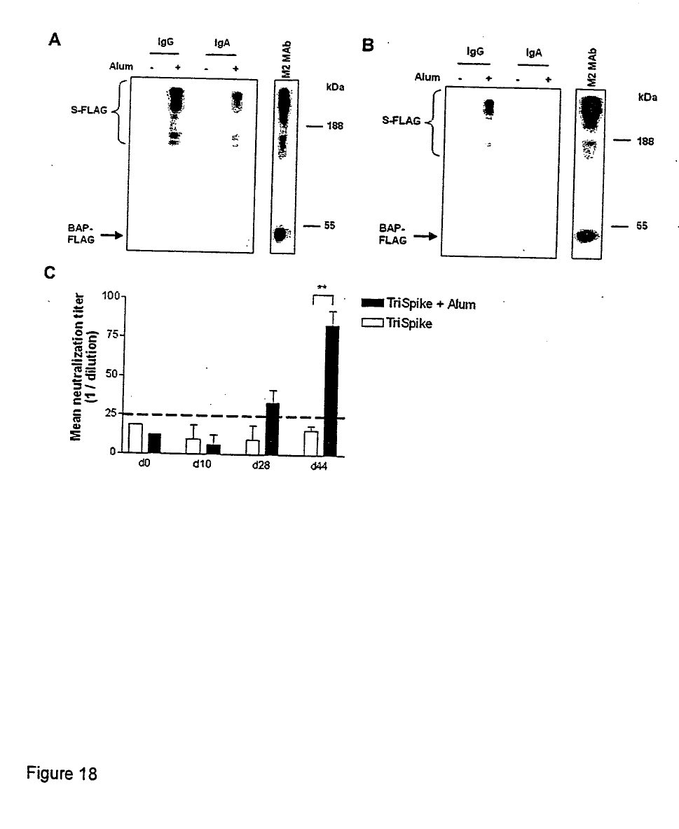

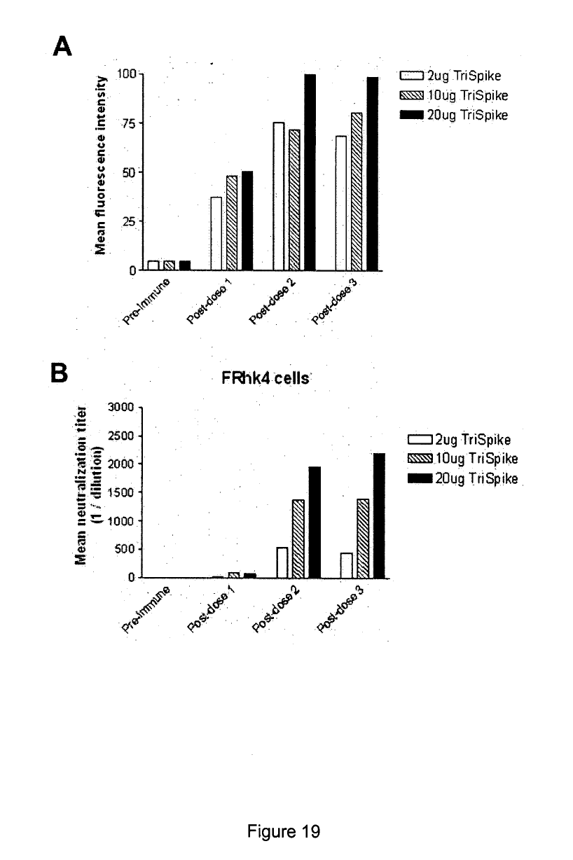

TriSpike in Alum adjuvant induced an enhanced mucosal IgG and IgA response. SARS CoV can be detected in the upper and lower respiratory tract of humans and infected laboratory animals. In addition to the respiratory tract, SARS CoV can be detected in intestinal tissue of fatal cases (AJG, 2005, 100, 169-176). In order to study the capacity of the TriSpike candidate vaccine to induce SARS CoV specific IgG and IgA antibodies at mucosal sites, we collected fecal and nasal lavage samples from mice immunized with TriSpike±Alum adjuvant by the intraperitoneal route. Fecal samples were prepared as described previously (PNAS, 2004, 101, 13584-13589). Briefly, fecal pellets (˜100 mg) were collected on indicated days. Fecal extracts were prepared by adding 0.5 ml of PBS containing 0.02% Na-azide for 30 min at 40° C. with gentle rotation and cleared by centrifugation (13,000 rpm). Generally, 0.2 ml of clear supernatant could be obtained from one tube of fecal pellet suspension. Western blot analysis (FIG. 18) showed the presence of mucosal IgG and IgA response from fecal sample only in mice immunized with TriSpike+alum but not in mice immunized with TriSpike alone. Similarly, only Ig contained in the fecal sample from TriSpike+alum immunized mice showed neutralization activity against SARS CoV in micro-neutralization assay. The detection of fecal sample IgG and IgA from mice immunized with TriSpike+alum indicates the development of a first-line defense mechanism against SARS CoV infection within the gastrointestinal system of TriSpike immunized animals.

Nasal lavage samples were prepared as detailed in Current Protocols in Immunology (Chapter 19, 11.15-16). Briefly, nasal lavage samples were collected on indicated days. Anesthesia of mice was performed with twice the volume of ketamine/xylazine solution injected intraperitonically into naive or immunized mice. The thoracic cavity was opened and 25 G needle was inserted with 0.5 ml PBS/aprotinin injected into the tracheal lumen cephalic to the obstruction. About 0.5 ml of nasal wash sample could be collected from each mouse.

Western blot analysis of pooled nasal lavage sample (n=3) indicated the presence of mucosal IgG in the nasal lavage sample in TriSpike+alum immunization mice, but not in mice immunized with TriSpike alone. However, no IgA response was detected in nasal lavage samples of either TriSpike+alum or TriSpike alone immunization, which may result because of the route of antigen administration. The presence of mucosal IgG response from nasal sample did not produce any protective effect against SARS CoV infection in vitro based on the micro-neutralization assay result.

What is claimed is:

1. A purified nucleic acid molecule comprising SEQ ID NO: 2 (Spike-Pasteur-modif), SEQ ID NO: 3 (Spike-HKU-PRC), or SEQ ID NO: 6.

2. A purified nucleic acid molecule encoding an amino acid sequence comprising the sequence of SEQ ID NO: 4 or SEQ ID NO: 7, wherein said purified nucleic acid molecule shows increased expression of Spike protein as compared to SEQ ID NO: 1.

3. A purified nucleic acid molecule that hybridizes to either strand of a denatured, double-stranded DNA comprising the nucleic acid sequence of SEQ ID NO: 3 or SEQ ID NO: 6 under conditions of high stringency.

4. The purified nucleic acid molecule of claim 3, wherein said purified nucleic acid molecule shows increased expression of Spike protein as compared to SEQ ID NO:1.

5. The purified nucleic acid molecule of claim 4 comprising a substitution of at least one negative cis-acting signal, and wherein the encoded polypeptide sequence of said Spike protein remains unchanged.

6. The purified nucleic acid molecule of claim 5, wherein said negative cis-acting signal comprises at least one of the following:
(a) an AU-rich RNA instability motif;
(b) a repeating sequence;
(c) a secondary stretch;
(d) a splice donor and acceptor site; and
(e) an internal poly(A) site.

7. The purified nucleic acid molecule of claim 6, wherein said purified nucleic acid molecule further comprises at least one additional expression enhancing sequence.

8. The purified nucleic acid molecule of claim 7, wherein said additional expression enhancing sequence comprises at least one of the following:
(a) a Kozak consensus sequence; and
(b) an additional STOP codon.

9. The purified nucleic acid molecule of claim 4, wherein codon usage has been optimized to the bias of Cricetulus griseus.

10. The purified nucleic acid molecule of claim 4, wherein the portion of said purified nucleic acid molecule encoding said Spike protein comprises at least about a 10 percent increase in the percentage GC-content as compared to SEQ ID NO: 1.

11. The purified nucleic acid molecule of claim 7, wherein said substitution of at least one negative cis-acting signal and wherein said at least one additional expression enhancing sequence does not include the following:
(a) internal TATA-boxes, chi-sites, and ribosomal entry sites;
(b) AT-rich or GC-rich sequence stretches;
(c) repeat sequences and RNA secondary structures; and
(d) splice donor and acceptor sites.

12. A recombinant vector that directs the expression of a nucleic acid molecule selected from the group consisting of the purified nucleic acid molecules of claims 1-3.

13. A purified polypeptide comprising SEQ ID NO: 4, SEQ ID NO: 5, or SEQ ID NO: 7.

14. A purified polypeptide encoded by a nucleic acid molecule selected from the group consisting of the purified nucleic acid molecules of claims 1-3.

15. The purified polypeptide of claim 14, wherein said purified polypeptide comprises high mannose EndoH sensitive N-glycans.

16. Purified antibodies that bind to a polypeptide of claim 14.

17. Purified antibodies of claim 16, wherein said antibodies are monoclonal antibodies.

18. Purified antibodies of claim 16, wherein said antibodies comprise neutralizing antibodies.

19. A host cell transfected or transduced with the vector of claim 12.

20. The host cell of claim 19, wherein said host cell is selected from the group consisting of 293T cells, BHK cells, and FRHK4 cells.

21. A method for improving the expression of SARS CoV Spike polypeptide by a nucleic acid molecule comprising reducing the number of negative cis-acting signals in the nucleic acid molecule, wherein the reduction in the number of negative cis-acting signals occurs without altering the sequence of said SARS CoV Spike polypeptide.

22. The method of claim 21, wherein said negative cis-acting signals comprise at least one of the following: an AU-rich RNA instability motif; a repeating sequence; secondary stretches; a splice donor and acceptor site; and an internal poly(A) site.

23. The method of claim 22, wherein said method further comprises introducing additional signals without altering the sequence of said SARS CoV Spike polypeptide.

24. The method of claim 23, wherein said additional signals comprises at least one of a Kozak consensus sequence and an additional STOP codon.

25. The method of claim 24, further comprising optimizing codon usage to the bias of Cricetulus griseus.

26. The method of claim 24, further comprising increasing GC-content within the coding region of the Spike nucleotide at least about 10%.

27. An isolated immunological complex comprising a SARS CoV Spike polypeptide and an antibody that specifically recognizes said polypeptide.

28. An isolated immunological complex comprising a SARS CoV Spike polypeptide and an antibody that specifically recognizes said polypeptide, wherein said antibody is raised against the purified polypeptide of claim 14.

29. A method for detecting infection by SARS virus, wherein the method comprises providing a composition comprising a biological material suspected of being infected with SARS virus, and assaying for the presence of Spike polypeptide.

30. The method of claim 29, wherein said Spike polypeptide is assayed by electrophoresis or by immunoassay with antibodies that are immunologically reactive with the Spike polypeptide.

31. A method for detecting infection by SARS virus, wherein the method comprises providing a composition comprising a biological material suspected of being infected with SARS virus, and assaying for the presence of Spike polypeptide, wherein said antibodies were raised against the purified polypeptide of claim 14.

32. An in vitro diagnostic method for the detection of the presence or absence of antibodies, which bind to an antigen comprising SARS CoV Spike polypeptide, wherein the method comprises contacting the antigen with a biological fluid for a time and under conditions sufficient for the antigen and antibodies in the biological fluid to form an antigen-antibody complex, and detecting the formation of the complex.

33. The method of claim 32, which further comprises measuring the formation of the antigen-antibody complex.

34. The method of claim 33, wherein the formation of antigen-antibody complex is detected by immunoassay based on Western blot technique, ELISA, indirect immunofluorescence assay, or immunoprecipitation assay.

35. An in vitro diagnostic method for the detection of the presence or absence of antibodies, which bind to an antigen comprising the purified polypeptide of claim 14, wherein the method comprises contacting the antigen with a biological fluid for a time and under conditions sufficient for the antigen and antibodies in the biological fluid to form an antigen-antibody complex, and detecting the formation of the complex.

36. A diagnostic kit for the detection of the presence or absence of antibodies, which bind to SARS CoV Spike polypeptide or mixtures thereof, wherein the kit comprises an antigen comprising SARS CoV Spike polypeptide or mixtures of SARS CoV Spike polypeptides, and means for detecting the formation of immune complex between the antigen and antibodies, wherein the means are present in an amount sufficient to perform said detection.

37. A diagnostic kit for the detection of the presence or absence of antibodies, which bind to SARS CoV Spike polypeptide or mixtures thereof, wherein the kit comprises an antigen comprising the purified polypeptide of claim 14, and means for detecting the formation of immune complex between the antigen and antibodies, wherein the means are present in an amount sufficient to perform said detection.

38. An immunogenic composition comprising at least one SARS CoV Spike polypeptide in an amount sufficient to induce an immunogenic or protecting response in vivo, and a pharmaceutically acceptable carrier therefor.

39. The immunogenic composition of claim 38, wherein said composition comprises a neutralizing amount of at least one SARS CoV Spike polypeptide.

40. The immunogenic composition of claim 38, further comprising an Alum adjuvant.

41. An immunogenic composition comprising the purified polypeptide of claim 14, in an amount sufficient to induce an immunogenic or protecting response in vivo, and a pharmaceutically acceptable carrier therefor.

42. A method of treating a host with the immunogenic composition of claim 41, comprising administering said immunogenic composition to the host in an amount sufficient to induce an immunogenic or protecting response in vivo.

43. The method of claim 42, wherein the immunogenic composition is administered by the intraperitoneal route.

44. The method of claim 42, wherein the immunogenic composition further comprises Alum adjuvant.

45. The method of claim 42, wherein the immunogenic composition is administered in a dosage regimen comprising two or more administrations of 2-20 μg of SARS CoV Spike peptide.

46. A vaccine composition against SARS CoV comprising the polypeptide of claim 14.

47. A method of vaccinating against SARS CoV comprising administering to an animal in need thereof the vaccine composition of claim 46.

48. The method of claim 47, wherein the method of vaccinating induces enhanced mucosal IgA and IgG antibodies.

49. The method of claim 48, wherein the vaccine composition further comprises Alum adjuvant.

50. The method of claim 49, wherein the vaccine composition is administered in a dosage regimen comprising two or more administrations of 2-20 μg of SARS CoV Spike peptide with Alum adjuvant.

51. A method for detecting the presence or absence of SARS CoV comprising:
(1) contacting a sample suspected of containing viral genetic material of SARS CoV with at least one nucleotide probe, and
(2) detecting hybridization between the nucleotide probe and the viral genetic material in the sample, wherein said nucleotide probe is complementary to the full-length sequence of the purified nucleic acid of SEQ ID NO: 1, SEQ ID NO: 2, SEQ ID NO: 3, or SEQ ID NO: 6.

52. A plasmid deposited at C.N.C.M having the accession number I-3221, I-3222, or I-3223.

53. A SARS CoV Spike polypeptide encoded by a plasmid of claim 52.

54. A polynucleotide encoding a fragment of the SARS CoV Spike polypeptide having at least one mutation compared with SEQ ID NO: 1, SEQ ID NO: 2, SEQ ID NO: 3, or SEQ ID NO: 6.

55. A fragment of the nucleotide sequence or a polynucleotide according to claim 54 comprising at least 10 continuous nucleotides and a maximum of 150 continuous nucleotides.

56. A composition of polynucleotides comprising at least the nucleotide sequence of claims 54 or 55.

57. A polypeptide or a polynucleotide according to any one of claims 1-11, 13-15, and 53-55 capable of inducing a T-cell response against a SARS infection.


SEQUENCE LISTING (7 sequences)

SEQ ID NO: 1 - DNA, 3795 bp, SARS Coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtctctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaatccatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatgggcttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggctaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgacgctgagccagttctcaagggtgtcaaattacattacacagactacaaggatgacgatgacaataagtaa

SEQ ID NO: 2 - DNA, 3795 bp, SARS Coronavirus
atgtttattttcttattatttcttactctcacgagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtctctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaatccatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatgggcttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggctaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgacgagttgttgcagttgcctcaagggtgcatgctcttgtggttcttgctgcaagtttgatgaggatgacgctgagccagttctcaagggtgtcaaattacattacacagactacaaggatgacgatgacaataagtaa

SEQ ID NO: 3 - DNA, 3897 bp, SARS Coronavirus
  CDS (44)..(3844)
ctatagggcgaattgggtaccgctagcggatccgcgcgccaccatgtttattttcctgctgtttctgactctgaccagcggcagtgacctggaccggtgcaccacttttgatgatgtgcaggctcctaattacactcagcatacttcctctatgaggggcgtgtactatcctgatgaaatttttagatccgacactctgtatctgactcaggatctgtttctgccattctattctaatgtgacaggctttcatactattaatcatacctttggcaaccctgtgatcccttttaaggatggcatctattttgctgccacagagaagtccaatgtggtgcggggatgggtgttcggctctaccatgaacaacaagtcccagtccgtgattattattaacaattctactaatgtggtgatccgagcctgtaactttgaactgtgtgacaacccattctttgctgtgtctaagcccatgggcacacagacacatactatgatcttcgataatgcctttaattgcactttcgagtacatctctgatgccttttccctggatgtgtccgaaaagtccggcaactttaagcacctgcgagagtttgtgtttaagaataaggatggctttctgtatgtgtataagggctatcagcctatcgacgtggtgcgcgatctgccttctggctttaacactctgaagcctatttttaagctgcctctgggcattaacattacaaattttcgggccattctgacagcctttagccctgctcaggacatttggggcacctctgctgccgcctattttgtgggctatctgaagccaactacctttatgctgaagtatgatgaaaatggcacaatcacagatgctgtggattgttctcagaatccactggctgaactgaagtgctctgtgaagagctttgagattgacaagggaatctaccagacctctaatttccgcgtggtgccctctggagatgtggtgagattccctaatattacaaacctgtgtccttttggagaagtgtttaatgctactaagttcccttctgtgtatgcctgggagagaaagaagatttctaattgtgtggctgattactctgtgctgtacaactccacattttttagcacctttaagtgctatggcgtgtctgccactaagctgaatgatctgtgcttctccaatgtgtatgccgattcttttgtggtgaagggagatgatgtgagacagatcgccccaggacagactggcgtgattgctgattacaattataagctgccagatgatttcatgggctgtgtgctggcttggaatactaggaacattgatgctacttccactggcaattataattacaagtatcggtatctgagacatggcaagctgaggccctttgagagagacatctctaacgtgcctttcagccctgatggcaagccttgcaccccacctgctctgaattgttattggccactgaatgattatggcttttacaccactactggcattggctaccagccttacagagtggtggtgctgtcttttgaactgctgaatgcccctgccacagtgtgtggaccaaagctgtccactgacctgattaagaaccagtgtgtgaactttaactttaatggactgactggcactggcgtgctgactccttctagcaagagatttcagccatttcagcagtttggccgggatgtgtctgatttcactgattccgtgcgagatcctaagacatctgaaatcctggacatttccccttgctcttttggcggcgtgagcgtgattacacctggaacaaatgcttcctctgaagtggctgtgctgtatcaggatgtgaactgcactgatgtgtctacagccatccatgccgatcagctgacaccagcttggcgcatctattctactggaaacaatgtgttccagactcaggccggctgtctgatcggagctgagcatgtggacacttcttatgagtgcgacattcctattggagctggcatttgtgctagttaccatacagtgtctctgctgcggagtactagccagaagtctattgtggcttatactatgtctctgggcgctgatagttccattgcttactctaataacaccattgctatccctactaacttttccattagcattactacagaagtgatgcctgtgtctatggctaagacctccgtggattgtaatatgtacatctgcggagattctaccgaatgtgctaatctgctgctgcagtatggcagcttttgcacacagctgaatcgggctctgtctggcattgctgctgaacaggatcgcaacacacgggaagtgttcgctcaagtgaagcagatgtataagaccccaactctgaagtattttggcggctttaatttttcccagatcctgcctgaccctctgaagcccactaagcggtcttttattgaggacctgctgtttaacaaagtgacactggctgatgctggctttatgaagcagtatggcgaatgcctgggcgatattaatgctagagatctgatttgtgcccagaagttcaatggcctgacagtgctgcctcctctgctgactgatgatatgattgctgcctacactgctgctctggtgtctggcactgccactgctggatggacatttggcgctggcgctgctctgcagatcccttttgctatgcagatggcctatcggttcaatggcattggagtgacccagaatgtgctgtatgagaaccagaagcagattgccaaccagtttaacaaggccattagtcagattcaggaatccctgacaacaacatccactgccctgggcaagctgcaggacgtggtgaaccagaatgctcaggccctgaacacactggtgaagcagctgagcagcaattttggcgccatttccagtgtgctgaatgatatcctgtcccgactggataaagtggaggccgaagtgcagattgacaggctgattacaggcagactgcagagcctgcagacctatgtgacacagcagctgatcagggctgctgaaatcagggcttctgccaatctggctgctactaagatgtctgagtgtgtgctgggacagtccaagagagtggacttttgtggaaagggctaccacctgatgtccttcccacaggctgcccctcatggagtggtgttcctgcatgtgacctatgtgccatcccaggagaggaacttcaccacagccccagccatttgtcatgaaggcaaggcctacttccctcgggaaggcgtgttcgtgtttaatggcacttcttggtttattacacagcggaacttctttagcccacagatcatcactacagacaatacatttgtgtccggaaattgtgatgtggtgattggcatcattaacaacacagtgtatgatcctctgcagcctgagctggactccttcaaggaagagctggacaagtacttcaagaatcatacatccccagatgtggatctgggcgacatttccggcattaacgcttctgtggtgaacattcagaaggaaattgaccgcctgaatgaagtggctaagaatctgaatgaatccctgattgacctgcaggaactgggcaagtatgagcagtatattaagtggccttggtatgtgtggctgggcttcattgctggactgattgccatcgtgatggtgacaatcctgctgtgttgcatgacctcctgttgcagttgcctgaagggcgcttgctcttgtggatcttgctgcaagtttgatgaggatgactctgagccagtgctgaagggcgtgaagctgcattacacagggcccggcggcgactacaaggacgatgacgacaagtgatagatcgatgcatggatccgtttaaaccgagctccagctttgttccctta

SEQ ID NO: 4 - protein, 1267 aa, SARS Coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYTGPGGDYKDDDDK

SEQ ID NO: 5 - protein, 1267 aa, SARS Coronavirus; identical to SEQ ID NO: 4

SEQ ID NO: 6 - DNA, 3765 bp, SARS Coronavirus
atgtttattttcctgctgtttctgactctgaccagcggcagtgacctggaccggtgcaccacttttgatgatgtgcaggctcctaattacactcagcatacttcctctatgaggggcgtgtactatcctgatgaaatttttagatccgacactctgtatctgactcaggatctgtttctgccattctattctaatgtgacaggctttcatactattaatcatacctttggcaaccctgtgatcccttttaaggatggcatctattttgctgccacagagaagtccaatgtggtgcggggatgggtgttcggctctaccatgaacaacaagtcccagtccgtgattattattaacaattctactaatgtggtgatccgagcctgtaactttgaactgtgtgacaacccattctttgctgtgtctaagcccatgggcacacagacacatactatgatcttcgataatgcctttaattgcactttcgagtacatctctgatgccttttccctggatgtgtccgaaaagtccggcaactttaagcacctgcgagagtttgtgtttaagaataaggatggctttctgtatgtgtataagggctatcagcctatcgacgtggtgcgcgatctgccttctggctttaacactctgaagcctatttttaagctgcctctgggcattaacattacaaattttcgggccattctgacagcctttagccctgctcaggacatttggggcacctctgctgccgcctattttgtgggctatctgaagccaactacctttatgctgaagtatgatgaaaatggcacaatcacagatgctgtggattgttctcagaatccactggctgaactgaagtgctctgtgaagagctttgagattgacaagggaatctaccagacctctaatttccgcgtggtgccctctggagatgtggtgagattccctaatattacaaacctgtgtccttttggagaagtgtttaatgctactaagttcccttctgtgtatgcctgggagagaaagaagatttctaattgtgtggctgattactctgtgctgtacaactccacattttttagcacctttaagtgctatggcgtgtctgccactaagctgaatgatctgtgcttctccaatgtgtatgccgattcttttgtggtgaagggagatgatgtgagacagatcgccccaggacagactggcgtgattgctgattacaattataagctgccagatgatttcatgggctgtgtgctggcttggaatactaggaacattgatgctacttccactggcaattataattacaagtatcggtatctgagacatggcaagctgaggccctttgagagagacatctctaacgtgcctttcagccctgatggcaagccttgcaccccacctgctctgaattgttattggccactgaatgattatggcttttacaccactactggcattggctaccagccttacagagtggtggtgctgtcttttgaactgctgaatgcccctgccacagtgtgtggaccaaagctgtccactgacctgattaagaaccagtgtgtgaactttaactttaatggactgactggcactggcgtgctgactccttctagcaagagatttcagccatttcagcagtttggccgggatgtgtctgatttcactgattccgtgcgagatcctaagacatctgaaatcctggacatttccccttgctcttttggcggcgtgagcgtgattacacctggaacaaatgcttcctctgaagtggctgtgctgtatcaggatgtgaactgcactgatgtgtctacagccatccatgccgatcagctgacaccagcttggcgcatctattctactggaaacaatgtgttccagactcaggccggctgtctgatcggagctgagcatgtggacacttcttatgagtgcgacattcctattggagctggcatttgtgctagttaccatacagtgtctctgctgcggagtactagccagaagtctattgtggcttatactatgtctctgggcgctgatagttccattgcttactctaataacaccattgctatccctactaacttttccattagcattactacagaagtgatgcctgtgtctatggctaagacctccgtggattgtaatatgtacatctgcggagattctaccgaatgtgctaatctgctgctgcagtatggcagcttttgcacacagctgaatcgggctctgtctggcattgctgctgaacaggatcgcaacacacgggaagtgttcgctcaagtgaagcagatgtataagaccccaactctgaagtattttggcggctttaatttttcccagatcctgcctgaccctctgaagcccactaagcggtcttttattgaggacctgctgtttaacaaagtgacactggctgatgctggctttatgaagcagtatggcgaatgcctgggcgatattaatgctagagatctgatttgtgcccagaagttcaatggcctgacagtgctgcctcctctgctgactgatgatatgattgctgcctacactgctgctctggtgtctggcactgccactgctggatggacatttggcgctggcgctgctctgcagatcccttttgctatgcagatggcctatcggttcaatggcattggagtgacccagaatgtgctgtatgagaaccagaagcagattgccaaccagtttaacaaggccattagtcagattcaggaatccctgacaacaacatccactgccctgggcaagctgcaggacgtggtgaaccagaatgctcaggccctgaacacactggtgaagcagctgagcagcaattttggcgccatttccagtgtgctgaatgatatcctgtcccgactggataaagtggaggccgaagtgcagattgacaggctgattacaggcagactgcagagcctgcagacctatgtgacacagcagctgatcagggctgctgaaatcagggcttctgccaatctggctgctactaagatgtctgagtgtgtgctgggacagtccaagagagtggacttttgtggaaagggctaccacctgatgtccttcccacaggctgcccctcatggagtggtgttcctgcatgtgacctatgtgccatcccaggagaggaacttcaccacagccccagccatttgtcatgaaggcaaggcctacttccctcgggaaggcgtgttcgtgtttaatggcacttcttggtttattacacagcggaacttctttagcccacagatcatcactacagacaatacatttgtgtccggaaattgtgatgtggtgattggcatcattaacaacacagtgtatgatcctctgcagcctgagctggactccttcaaggaagagctggacaagtacttcaagaatcatacatccccagatgtggatctgggcgacatttccggcattaacgcttctgtggtgaacattcagaaggaaattgaccgcctgaatgaagtggctaagaatctgaatgaatccctgattgacctgcaggaactgggcaagtatgagcagtatattaagtggccttggtatgtgtggctgggcttcattgctggactgattgccatcgtgatggtgacaatcctgctgtgttgcatgacctcctgttgcagttgcctgaagggcgcttgctcttgtggatcttgctgcaagtttgatgaggatgactctgagccagtgctgaagggcgtgaagctgcattacaca

SEQ ID NO: 7 - protein, 1255 aa, SARS Coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT